(19) United States
(12) Patent Application Publication
(10) Pub. No.: US 2008/0152694 A1
(43) Pub. Date: Jun. 26, 2008

(54) Devices, Systems and Methods for Ophthalmic Drug Delivery

(75) Inventors: Thomas J. Lobl, Valencia, CA (US); Anna Imola Nagy, Valencia, CA (US); Jacob E. Pananen, Pasadena, CA (US); John V. Schloss, Saugus, CA (US)

Correspondence Address: BANNER & WITCOFF, LTD., 1100 13th STREET, N.W., SUITE 1200, WASHINGTON, DC, 20005-4051, US

(73) Assignee: NEUROSYSTEC CORPORATION, Valencia, CA (US)

(21) Appl. No.: 11/780,853

(22) Filed: Jul. 20, 2007

Related U.S. Application Data
(60) Provisional application No. 60/807,900, filed on Jul. 20, 2006.

(51) Int. Cl. A61K 9/00; A61K 9/14; A61K 31/4523; A61P 27/02

(52) U.S. Cl. 424/427; 424/489; 514/326

(57) ABSTRACT

Devices, systems and techniques for delivering drugs to an ocular tissue are described. In at least some embodiments, a terminal component (e.g., a needle or open end of a catheter) is implanted in an ocular tissue and used to deliver one or more drugs. The delivered drugs may come from a source which is also implanted, or may be introduced from an external source (e.g., via a port). Both solid and liquid drug formulations can be used. Ocular implants can alternatively include a thin film coating that releases a drug into an ocular tissue.

### CROSS-REFERENCE TO RELATED APPLICATIONS

This application claims the benefit of U.S. Provisional Application Ser. No. 60/807,900 (attorney docket number 006501.00023), filed Jul. 20, 2006 and titled “Devices, Systems and Methods for Ophthalmic Drug Delivery,” hereby incorporated by reference herein.

### BACKGROUND OF THE INVENTION

It is well known that drugs work most efficiently in the body of a human or animal if they are delivered locally where needed. When delivered systemically there is a much greater chance for side effects, as all tissues are exposed to large quantities of the drug. However, if the affected area is inside the body, localized drug delivery presents challenges. Local delivery to tissues located in anatomically difficult areas often requires a specialized injection device. This is especially true for injections into the eye.

Many treatments of ocular diseases rely on topical application of solutions (in drops) to the surface of the eye. The usefulness of topical drug application is limited by the significant flux barrier provided by the corneal epithelium and the rapid and extensive pre-corneal loss that occurs as a result of drainage and tear fluid turnover. It has been estimated that typically less than 5% of a topically applied drug permeates the cornea.

Although delivery of high concentrations of drugs as topical formulations has proven to be effective, the delivery of therapeutic doses of drugs to the tissues in the posterior segment of the eye remains a significant challenge. There are numerous diseases affecting the posterior segment, including age-related macular degeneration, diabetic retinopathy, glaucoma, and retinitis pigmentosa. Intravitreal injections provide the most direct approach to delivering drugs to the tissues of the posterior segment and for achieving therapeutic tissue drug levels. However, repeat injections are often required. Most patients would find such injections to be quite unpleasant. Repeat injections may also cause side effects such as retinal detachment, hemorrhage, endophthalmitis and cataract. Repeat injections also increase the potential for infections.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In at least some embodiments, a device for delivering drugs to the eye includes components such as a pump, filters and a fluid carrying system. Devices according to at least some embodiments can be used to deliver multiple bolus doses or continuous infusions of drugs to the eye over a longer period of time such as, but not limited to, a few days.

Some embodiments of the invention include implantable drug delivery systems which can be used for targeted delivery of drugs to the eye. Using such systems, small volumes of drugs can be delivered to the eye, either intermittently or continuously, on a short-term or a long-term (e.g., several months or years) basis. In some embodiments, an implanted osmotic pump contains solid or liquid drug (or is in fluid communication with a drug/filter capsule) and delivers drug through a catheter and a needle or other terminal component implanted in an eye.

Both solid and liquid drug formulations can be used. In embodiments using solid drugs, a separate drug vehicle can be used to entrain a portion of a solid drug mass contained in port reservoir or a drug-holding capsule. Examples of vehicles include, but are not limited to, saline, Ringer's solution, Ringer's lactate, artificial vitreous humor, and/or any other vehicle compatible with injection into the anterior chamber and/or posterior segment of the eye or otherwise into ocular tissue. The vehicle is then delivered to the eye or other ocular tissue via an implanted catheter.

### BRIEF DESCRIPTION OF THE DRAWINGS

The foregoing summary of the invention, as well as the following detailed description of certain embodiments, is better understood when read in conjunction with the accompanying drawings, which are included by way of example and not by way of limitation.

FIG. 1 is a drawing of an implantable drug delivery system, according to at least some embodiments, that includes an osmotic pump and solid drug/filter housing.

FIG. 2 is a cross-sectional view of the solid drug/filter housing shown in FIG. 1.

FIGS. 3A and 3B show an implantable drug delivery system according to at least some additional embodiments.

FIG. 4 shows a drug delivery device according to another embodiment.

FIG. 5 is a cross-sectional view of a sleeved drug reservoir from FIG. 4.

FIGS. 6A through 6C are cross-sectional views of drug reservoirs including screens.

FIGS. 6D and 6E are perspective and cross-sectional views, respectively, of a drug reservoir that includes an air vent.

FIG. 6F is a perspective view of a drug reservoir that includes flats.

FIG. 7 is a cross sectional view of a solid drug and 3-D antibacterial filter housing.

FIGS. 8 and 9 show a two piece solid drug and 3-D antibacterial filter housing according to another embodiment.

FIG. 10 shows an embodiment in which a dual lumen tube extends from a pump and/or reservoir containing solid drug.

FIG. 11 is an enlarged view of the distal ends of the dual lumen tube shown in FIG. 10.

FIG. 12 is a perspective view showing an embodiment in which a semi-permeable membrane allows interstitial fluid to pass into a chamber containing a solid drug.

FIG. 13 is a fully cross-sectional view of the embodiment of FIG. 12.

FIG. 14 shows the embodiment of FIGS. 12 and 13 containing solid drug pellets.

FIG. 15 shows an embodiment where fluid is circulated unidirectionally through a loop containing a semi-permeable hollow fiber.

FIGS. 16 through 19 show an embodiment implementing electrophoresis-stimulated delivery of drug.

FIG. 20 is a drawing of a port, catheter and terminal component.

FIG. 21 shows a subcutaneously-implantable port attached with a catheter to a sleeved drug reservoir.

FIG. 22 shows an ocular implant with a thin film coating according to at least some embodiments.

FIGS. 23 and 24 show a retinal implant according to at least some embodiments.

FIGS. 25 and 26 show examples of locations within an eye where terminal components according to certain embodiments may be implanted.

FIG. 27 shows the elution of gacyclidine from a drug dissolution chamber as a function of the concentration of hydrochloric acid in Ringer's solution used to erode pellets of crystalline gacyclidine base.

### DETAILED DESCRIPTION

### Overview

Described herein are at least some embodiments of systems, devices and methods for ophthalmic delivery of drugs, i.e., to delivery of drugs to ocular tissue(s). As used herein, “ocular tissue” refers to the eye, including tissues within the sclera (e.g., the retina) and outside the sclera (e.g., ocular muscles within the orbit). “Ocular tissue” also includes tissues neurologically connected to (but distinct from) the eye, such as the optic nerve, the geniculate nucleus and the visual cortex. Some embodiments include a subcutaneous pump (such as an osmotic pump) and reservoir attached to a catheter. A terminal component is attached to (or is part of) the catheter and is the element from which a drug is released into the eye. In some cases, a terminal component is a soft tissue catheter (e.g., a small-diameter flexible polymeric tube made from, e.g., polyimide, a fluoropolymer, silicone, polyurethane or PVC) which passes through an incision in the sclera and injects fluid into specific regions within the inner eye. In some embodiments (e.g., short term treatment of an acute condition), the terminal component may be a needle. Depth and location of insertion of a terminal component depends on which region is being targeted in the eye or other ocular tissue. The catheter or needle may have an insertion stop which controls the depth of insertion. In most cases, the terminal component may be implanted so as to minimize interference with eye movement. One possible location for an incision to insert a terminal component is in the pars plana. Possible locations for terminating a catheter for drug delivery may be in the vitreous or in the anterior chamber, allowing drugs to be delivered in controlled doses to a precise area of the eye. The terminal end of the catheter may be fixed, for example via suture, surgical tack, a tissue adhesive, or a combination thereof, to tissue near the outer surface of the eye. When attached, the catheter does not affect or otherwise restrict movement of the eye. The pump may be secured in a cavity that has been drilled out by a physician. Such a cavity may be located under the scalp on the mastoid bone or in another location closer to the eye. A drug delivering catheter may lead to the eye or other ocular tissue through a hole drilled in the bone next to the eye.

A terminal component can be implanted within the eyeball or in locations outside the sclera (e.g., behind the eyeball but within the orbit). In some embodiments, most or all of the injection device (including an osmotic pump or other type of fluid moving device) is implanted. Various embodiments could include implantation of all of the injection device in conjunction with a retinal implant. A drug delivery catheter from a pump and reservoir could be bundled together with the wires from an electronic package for a retinal implant so as to avoid the necessity of a second puncture of the eyeball to deliver a desired amount of drug. If desired, however, a terminal component could be installed in one location and a retinal implant installed in a different location within the same eye.

In treating disorders of the optic nerve or neurological pathways from the retina to the visual cortex, the terminal component may be placed in a location between the retina and visual cortex, such as the geniculate nucleus, or in the visual cortex itself. Placement of the drug-releasing terminal component will depend on the tissue in greatest need of treatment and can differ from one patient to another. Placement outside of the eye could be to deliver drugs to the optic nerve or neurons involved in vision that have been affected by diseases or injury (such as trauma, including surgical trauma), vein occlusion or ischemia, diabetic neuropathy, or neurodegeneration due to other causes.

In some embodiments, the drug delivery system may be combined with another type of ocular electrode, with another type of retinal vision prosthesis, etc. As with a retinal implant, a drug delivery catheter could be bundled with the wires from an electronics package for the electrode or other device, thereby minimizing trauma to the eye and enabling delivery of drug near the co-implanted device.

The following description is generally organized into several parts. Part I generally discusses at least some of the ocular conditions that can be treated according to various embodiments, as well as examples of drugs that can be used. Part II generally discusses devices that can be used to deliver drugs to ocular tissue according to at least some embodiments. Several examples follow part II.

### Part I: Ocular Conditions and Drugs

Devices such as are described herein can be used to ameliorate numerous disorders affecting the eye. Such disorders include, but are not limited to, ocular infections, inflammatory diseases, neoplastic diseases, and degenerative disorders. Listed in Table 1 are some of the conditions which are believed to be treatable using systems, devices and/or methods such as are described herein.

TABLE 1

| Conditions | Non-Limiting Examples |
|---|---|
| degenerative disorders | dry macular degeneration, glaucoma, macular |
| | edema secondary to vascular disorders, |
| | retinitis pigmentosa and wet macular degeneration |
| inflammatory diseases | birdshot retinopathy, diabetic retinopathy, |
| | Harada's and Vogt-Koyanagi-Harada |
| | syndrome, iritis, multifocal choroiditis and |
| | panuveitis, pars planitis, posterior scleritis, |
| | sarcoidosis, retinitis due to systemic lupus |
| | erythematosus, sympathetic ophthalmia, |
| | subretinal fibrosis, uveitis syndrome and |
| | white dot syndrome |
| ocular disorders associated with | age-related macular degeneration, angioid |
| neovascularization | streaks, branch retinal vein occlusion, |
| | choroiditis, corneal trauma-related disorders, |
| | diabetes-related iris neovascularization, |
| | diabetic retinopathy, idiopathic choroidal |
| | neovascularization, pathologic myopia, retinal |
| | detachment, retinal tumors, retinopathy of |
| | prematurity and sickle cell retinopathy |
| ocular infections associated with the | cytomegalovirus retinitis, histoplasma |
| choroids, retina or cornea | retinochoroiditis, toxoplasma retinochoroiditis |
| | and tuberculous choroiditis |
| neoplastic diseases | abnormal tissue growth (in the retina, choroid, |
| | uvea, vitreous or cornea), choroidal |
| | melanoma, intraocular lymphoma (of the |
| | choroids, vitreous or retina), metastatic |
| | lesions, retinoblastoma, and vitreous seeding |
| | from retinoblastoma |
| trauma | trauma incident to accidental injury or to |
| | surgery (e.g., placement of an ocular implant), |
| | retinal damage resulting from exposure to |
| | laser or other intense light |

Drug delivery devices according to at least some embodiments can be used to deliver one or more drugs to a particular target site so as to treat one or more of the conditions listed in Table 1 and/or to treat other conditions. The drug can be in solid, liquid or gel form. As used herein, the term “drug” includes any natural or synthetic, organic or inorganic, physiologically or pharmacologically active substance capable of producing a localized or systemic prophylactic and/or therapeutic effect when administered to an animal or human. A drug includes (i) any active drug, (ii) any drug precursor or pro-drug that may be metabolized within an animal or human to produce an active drug, (iii) combinations of drugs, (iv) combinations of drug precursors, (v) combinations of a drug with a drug precursor, and (vi) any of the foregoing in combination with a pharmaceutically acceptable carrier, excipient(s), slowly-releasing delivery system or formulating agent. As used herein, the term “drug” also includes, but is not limited to, any of one or more of the substances listed in Table 2.

TABLE 2

| Substance | Non-Limiting Examples |
|---|---|
| anti-infective agent | antibiotic, anti-fungal agent, anti-viral agent |
| anti-inflammatory agent | interferon α, steroid |
| anti-migraine medication | IMITREX ® |
| autonomic drug | adrenergic agent, adrenergic blocking agent, |
| | anticholinergic agent, skeletal muscle relaxant |
| blood formation or blood coagulation | anti-anemia drug, anti-coagulant, coagulant, |
| modulating agent | hemorrhagic agent, thrombolytic agent |
| anti-secretory molecule | proton-pump inhibitors such as pantoprazole, |
| | lansoprazole and rabeprazole; muscarinic |
| | antagonists such as atropine and scopolamine |
| central nervous system agent | analgesic, anti-convulsant, antipyretic |
| anti-neoplastic agent | chlorambucil, cyclosporine, interferon, methotrexate |
| hormone or synthetic hormone | triamcinolone acetonide |
| immunomodulating agent | etanercept, immunosuppresant |
| inorganic or organic molecule having | taurine, gacyclidine |
| therapeutic and/or prophylactic value | |
| peptide | fibronectin fragments of 10-20 amino acids in |
| | length; peptides with the . . . -ArgGlyAsp- . . . |
| | cell adhesion motif for inhibiting retinal |
| | detachment |
| protein | antibody or antigen binding portion thereof, |
| | fibronectin |
| cholinergic (para sympathomemitics) | physiostigmine, carbachol |
| β-adreneregic blockers | timolol |
| adrenergic agents (α/β agonists) | apraclonidine |
| carbonic anhydrase inhibitors | dorzolamide |
| prostaglandin analogs | latanoprost, prostaglandin F_{2α} |
| anti-angiogenic (e.g., anti-vascular | RGD-containing analogs and derivatives, |
| endothelial growth factor, or anti- | angiostatin, endostatin |
| VEGF) factors | |
| neurotrophic agents | nerve growth factor (NGF), neurotrophin-3 |
| | (NT3), brain-derived neurotrophic factor |
| | (BDNF) |

Additional examples are provided herein.

Many ophthalmic diseases and disorders are associated with one or more of angiogenesis, inflammation and degeneration. To treat these and other disorders, devices according to at least some embodiments permit delivery of anti-angiogenic factors; anti-inflammatory factors; factors that retard cell degeneration, promote cell sparing, or promote cell growth; and combinations of the foregoing. Using devices described and/or information provided herein, and based on the indications of a particular disorder, one of ordinary skill in the art can administer any suitable drug (or combination of drugs), such as the drugs described herein, at a desired dosage.

Any suitable biologically active molecules (“BAMs”) may also be delivered according to the devices, systems, and methods of this invention. Such molecules include, but are not limited to, antibodies, cytokines, enzymes, hormones, lymphokines, neuroprotective agents, neurotransmitters, and neurotrophic factors, as well as active fragments and derivatives of the foregoing. At least four types of BAMs are contemplated for delivery using devices according to at least some embodiments: (1) anti-angiogenic factors (2) anti-inflammatory factors, (3) factors that retard cell degeneration (anti-apoptosis agents), promote cell sparing or promote cell growth and (4) neuroprotective agents.

Angiogenesis inhibitors are compounds that reduce or inhibit the formation of new blood vessels in a mammal, and may be useful in the treatment of certain ocular disorders associated with neovascularization. Examples of useful angiogenesis inhibitors include, but are not limited to, the substances listed in Table 3.

TABLE 3

| Substance | Non-Limiting Examples |
|---|---|
| antibodies (and antigen binding | antibodies (and antigen binding fragments |
| fragments thereof) and peptides that | thereof) and peptides that bind preferentially |
| bind preferentially to and block or | to and block or reduce the binding activity of |
| reduce binding activity | the αᵥβ₃ integrin found on tumor |
| | vascular epithelial cells |
| | VLA-4 or α4β1 activity such as is |
| | described in U.S. Pat. No. 6,596,752 |
| | Epidermal Growth Factor receptor |
| | (EGFR) |
| | Vascular Endothelial Growth Factor |
| | receptor (VEGF) |
| | Anti-Epidermial Growth Factor |
| | Anti-Fibroblast Growth Factor |
| COX-2 selective inhibitors | CELEBREX ® |
| fumagillin (including analogs such as | |
| AGM-1470) | |
| protein/peptide inhibitors of | angiostatin (a proteolytic fragment of |
| angiogenesis | plasminogen) including full length amino acid |
| | sequences of angiostatin, endostatin (a |
| | proteolytic fragment of collagen XVIII) |
| | including full length amino acid sequences of |
| | endostatin and bioactive fragments thereof, |
| | and analogs thereof |
| small molecules anti-angiogenic agents | thalidomide |
| Tyrosine kinase inhibitors | halofuginone, PD 173074 |

As used herein, “bioactive fragments” refer to portions of an intact protein that have at least 30%, at least 70%, or at least 90% of the biological activity of the intact proteins. “Analogs” refer to species and allelic variants of the intact protein, or amino acid replacements, insertions or deletions thereof that have at least 30%, at least 70%, or at least 90% of the biological activity of the intact protein.

Diabetic retinopathy is characterized by angiogenesis. At least some embodiments contemplate treating diabetic retinopathy by implanting devices delivering one or more anti-angiogenic factors either intraocularly, preferably in the vitreous, or periocularly, preferably in the sub-Tenon's region. It may also be desirable to co-deliver one or more neurotrophic factors either intraocularly, periocularly, and/or intravitreally.

Several cytokines including bioactive fragments thereof and analogs thereof have also been reported to have anti-angiogenic activity and thus may be delivered using devices according to one or more embodiments. Examples include, but are not limited to, IL-12 (which reportedly works through an IFN-γ-dependent mechanism) and IFN-α (which has been shown to be anti-angiogenic alone or in combination with other inhibitors). The interferons IFN-α, IFN-β and IFN-γ reportedly have immunological effects, as well as anti-angiogenic properties, that are independent of their anti-viral activities.

Anti-angiogenic factors contemplated for use in at least some embodiments include, but are not limited to, angiostatin, anti-integrins, bFGF-binding molecules, endostatin, heparinase, platelet factor 4, vascular endothelial growth factor inhibitors (VEGF-inhibitors) and vasculostatin. The use of VEGF receptors Flt and Flk is also contemplated. When delivered in the soluble form these molecules compete with the VEGF receptors on vascular endothelial cells to inhibit endothelial cell growth.

VEGF inhibitors contemplated for use in at least some embodiments include, but are not limited to, VEGF-neutralizing chimeric proteins such as soluble VEGF receptors. In particular, one set of examples includes VEGF-receptor-IgG chimeric proteins. Another VEGF inhibitor contemplated for use in at least some embodiments is antisense phosphorothioate oligodeoxynucleotides (PS-ODNs).

It is contemplated that useful angiogenesis inhibitors, if not already known, may be identified using a variety of assays well known and used in the art. Such assays include, for example, the bovine capillary endothelial cell proliferation assay, the chick chorioallantoic membrane (CAM) assay or the mouse corneal assay.

Uveitis involves inflammation. At least some embodiments contemplate treating uveitis by intraocular, vitreal or anterior chamber implantation of devices releasing one or more anti-inflammatory factors. Anti-inflammatory factors contemplated for use in at least some embodiments include, but are not limited to, alpha-interferon (IFN-α), antiflammins, beta-interferon (IFN-β), glucocorticoids and mineralocorticoids from adrenal cortical cells, interleukin-10 (IL-10) and TGF-β. Certain BAMs may have more than one activity. For example, it is believed that IFN-α and IFN-β may have activities as both anti-inflammatory molecules and as anti-angiogenic molecules.

Retinitis pigmentosa is characterized by retinal degeneration. At least some embodiments contemplate treating retinitis pigmentosa by intraocular or vitreal placement of devices secreting one or more neurotrophic factors.

Age-related macular degeneration (wet and dry) involves both angiogenesis and retinal degeneration. At least some embodiments contemplate treating this disorder by using one or more of the herein-described devices to deliver one or more neurotrophic factors intraocularly, preferably to the vitreous, and/or one or more anti-angiogenic factors intraocularly or periocularly, preferably periocularly, most preferably to the sub-Tenon's region.

Factors contemplated for use in retarding cell degeneration, promoting cell sparing, or promoting new cell growth are collectively referred to herein as “neurotrophic factors.” Neurotrophic factors contemplated for use in at least some embodiments include, but are not limited to, acidic fibroblast growth factor (aFGF), basic fibroblast growth factor (bFGF), bone morphogenic proteins (BMP-1, BMP-2, BMP-7, etc.), brain-derived neurotrophic factor (BDNF), cardiotrophin-1 (CT-1), ciliary neurotrophic factor (CNTF), cytokines (such as IL-6, IL-10, CDF/LIF, and IFN-β), EGF, the family of transforming growth factors (including, e.g., TGFβ-1, TGF β-2, and TGF β-3), glial cell line derived neurotrophic factor (GDNF), the hedgehog family (sonic hedgehog, indian hedgehog, and desert hedgehog, etc.), heregulins, insulin-like growth factor-1 (IGF-1), interleukin 1-β (IL1-β), neuregulins, neurotrophin 3 (NT-3), neurotrophin 4/5 (NT-4/5), neurturin, nerve growth factor (NGF), PDGF, TGF-alpha. The preferred neurotrophic factors are GDNF, BDNF, NT-4/5, neurturin, CNTF, and CT-1.

Use of modified, truncated, and mutein forms of the above-mentioned molecules is also contemplated in at least some embodiments. Further, use of active fragments of these growth factors (i.e., those fragments of growth factors having biological activity sufficient to achieve a therapeutic effect) is also contemplated. Also contemplated is use of growth factor molecules modified by attachment of one or more polyethylene glycol (PEG) or other repeating polymeric moieties. Use of combinations of these proteins and polycistronic versions thereof is also contemplated.

Glaucoma is characterized by increased ocular pressure and loss of retinal ganglion cells. Treatments for glaucoma contemplated in at least some embodiments include delivery of one or more neuroprotective agents that protect cells from excitotoxic damage. Such agents include, but are not limited to, cytokines, N-methyl-D-aspartate (NMDA) antagonists and neurotrophic factors. These agents may be delivered intraocularly, preferably intravitreally. Gacyclidine (GK11) is an NMDA antagonist and is believed to be useful in treating glaucoma and other diseases where neuroprotection would be helpful or where there are hyperactive neurons. Additional compounds with useful activity are D-JNK-kinase inhibitors.

The term “drug” includes neuroprotective agents, i.e., agents capable of retarding, reducing or minimizing the death of neuronal cells. Neuroprotective agents may be useful in the treatment of various disorders associated with neuronal cell death (e.g., diabetic retinopathy, glaucoma, macular degeneration (wet and dry), retinitis pigmentosa, etc.). Examples of neuroprotective agents that may be used in at least some embodiments include, but are not limited to, apoptosis inhibitors, cAMP elevating agents, caspase inhibitors, neurotrophic factors and NMDA antagonists (such as gacyclidine and related analogs). Exemplary neurotrophic factors include, but are not limited to, the following: Brain Derived Growth Factor and bioactive fragments and analogs thereof, cytokine-associated neurotrophic factors; Fibroblast Growth Factor and bioactive fragments and analogs thereof, Insulin-like Growth Factors (IGF) and bioactive fragments and analogs thereof (e.g., IGF-I and IGF-II); and Pigment Epithelium Derived Growth Factor and bioactive fragments and analogs thereof. Exemplary cAMP elevating agents include, but are not limited to, the following: 8-(4-chlorophenylthio)-adenosine-3′:5′-cyclic-monophosphate (CPT-cAMP), 8-bromo-cAMP, dibutyryl-cAMP and dioctanoyl-cAMP, cholera toxin, forskolin and isobutyl methylxanthine. Exemplary caspase inhibitors include, but are not limited to, the following: caspase-1 inhibitors (e.g., Ac-N-Me-Tyr-Val-Ala-Asp-aldehyde; SEQ ID NO:1); caspase-2 inhibitors (e.g., Ac-Val-Asp-Val-Ala-Asp-aldehyde; SEQ ID NO:2); caspase-3 inhibitors (e.g., Ac-Asp-Glu-Val-Asp-aldehyde; SEQ ID NO:3); caspase-4 inhibitors (e.g., Ac-Leu-Glu-Val-Asp-aldehyde; SEQ ID NO:4); caspase-6 inhibitors (e.g., Ac-Val-Glu-Ile-Asp-aldehyde; SEQ ID NO:5); caspase-8 inhibitors (e.g., Ac-Asp-Glu-Val-Asp-aldehyde; SEQ ID NO:6); and caspase-9 inhibitors (e.g., Ac-Asp-Glu-Val-Asp-aldehyde; SEQ ID NO:7). Each of the aforementioned caspase inhibitors can be obtained from Bachem Bioscience Inc., PA or Peptides International, Inc., Louisville, Ky.

Devices according to at least some embodiments may be useful in the treatment of a variety of other ocular disorders. For example, a drug delivery device may deliver an anti-infective agent, such as an antibiotic, anti-viral agent or anti-fungal agent, for the treatment of an ocular infection. Similarly, a device may deliver a steroid, for example, hydrocortisone, dexamethasone sodium phosphate or methylprednisolone acetate, for the treatment of an inflammatory disease of the eye. A device may be used to deliver a chemotherapeutic or cytotoxic agent, for example, methotrexate, chlorambucil, cyclosporine, or interferon, for the treatment of an ocular neoplasm. Furthermore, a device may be useful in delivering one or more drugs for the treatment of certain degenerative ocular disorders. Additional examples of such drugs include, but are not limited to, the substances listed in Table 4.

TABLE 4

| Substance | Non-Limiting Examples |
|---|---|
| adrenergic agonists | apraclonidine (e.g., IOPIDINE ®), brimonidine |
| | (e.g., ALPHGAN ®), dipivefrin (e.g., |
| | PROPINE ®), epinephrine (e.g., EPIFRIN ®) |
| anti-inflammatory drug | steroid (e.g., hydrocortisone, dexamethasone |
| | sodium phosphate or methylprednisolone |
| | acetate), indomethacin, naprosyn, VEGF |
| | antagonist for the treatment of macular edema |
| | secondary to certain retinal vascular disorders |
| carbonic anhydrase | acetazolamide (e.g., DIAMOX ®), |
| inhibitors | methazolamide (e.g., NEPTAZANE ®), |
| | dorzolamide (e.g., TRUSOPT ®), brinzolamide |
| | (e.g., AZOPT ®) |
| integrin antagonists | LFA-1, VLA-4, Mac-1, ICAM-1, ICAM-2, |
| | ICAM-3, VCAM antagonist, molecules |
| | described in U.S. Pat. No. 6,670,321 for the |
| | treatment of diabetic retinopathy |
| chemokine antagonists | MCP-1, MCP-5, MCP-3, MIP1α, CCR5, |
| | RANTES |
| selectin antagonists | E-selectin, P-selectin and L-selectin |

As used herein, an antagonist may comprise, without limitation, an antibody, an antigen binding portion of an antibody, a biosynthetic antibody binding site that binds a particular target protein (e.g., ICAM-1), or an antisense molecule that hybridizes in vivo to a nucleic acid encoding a target protein or a regulatory element associated therewith. An antagonist may also comprise a ribozyme, aptamer, or small molecule that binds to and/or inhibits a target protein (e.g., ICAM-1) or that binds to and/or inhibits, reduces or otherwise modulates expression of nucleic acid encoding a target protein (e.g., ICAM-1).

At least some embodiments may be useful for the treatment of ocular neovascularization, a condition associated with many ocular diseases and disorders and accounting for a majority of severe visual loss. For example, contemplated is treatment of retinal ischemia-associated ocular neovascularization, a major cause of blindness in diabetes and many other diseases; corneal neovascularization, which predisposes patients to corneal graft failure; and neovascularization associated with diabetic retinopathy, central retinal vein occlusion, and possibly age-related macular degeneration.

At least some embodiments may also be used to treat ocular symptoms resulting from diseases or conditions that have both ocular and non-ocular symptoms. Examples include, but are not limited to, AIDS-related disorders such as cytomegalovirus retinitis and disorders of the vitreous, pregnancy-related disorders such as hypertensive changes in the retina, and ocular effects of various infectious diseases (e.g., cyst cercosis, fungal infections, Lyme disease, opthalmonyiasis, parasitic disease, syphilis, toxocara canis, tuberculosis, etc.).

Drugs may be introduced into a cavity of the eye (or to other ocular tissues) either in pure form or as a formulation, for example, in combination with a pharmaceutically acceptable carrier or encapsulated within a release system. The drugs can be homogeneously or heterogeneously distributed within the release system. A variety of release systems may be useful in the practice of the invention, however, the choice of the appropriate system will depend upon rate of drug release required by a particular drug regime. Both non-degradable and degradable release systems can be used. Suitable release systems include polymers and polymeric matrices, non-polymeric matrices, or inorganic and organic excipients and diluents. Release systems may be natural or synthetic. However, synthetic release systems are preferred because generally they are more reliable, more reproducible and produce more defined release profiles. The release system material can be selected so that drugs having different molecular weights are released from a particular cavity by diffusion through or degradation of the material. Embodiments of the invention include drug release via diffusion or degradation using biodegradable polymers, bioerodible hydrogels and protein delivery systems.

Embodiments of the invention can be used to deliver drugs that are in solid or in liquid formulations. Frequently, a solid drug has the advantage of maintaining its stability for longer periods of time. Solid drugs also have a high drug to volume ratio and low surface area. If solid drug is used, properties of a vehicle can be used to control the rate at which drug is removed (whether by dissolution, elution, erosion or some other mechanism or combination of mechanisms) from one or more masses of solid drug, thereby offering a flexibility for modulating a concentration of drug that is delivered to an ocular tissue. As used herein (including the claims), a “vehicle” is a fluid medium used to remove solid drug from one or more masses of solid drug and/or to deliver the removed drug to an ocular tissue. A vehicle can be a bodily fluid such as interstitial fluid, an artificial fluid or a combination of bodily and artificial fluids, and may also contain other materials in addition to a drug being removed and/or delivered. A vehicle may contain such other materials in solution (e.g., NaCl in saline, a solution of an acid or base in water, etc.) and/or suspension (e.g., nanoparticles). Further examples of vehicles are included below.

Drug that is removed from a solid drug mass by a vehicle and retained in that vehicle is sometimes referred to herein as being entrained within (or by) the vehicle. As used herein (including the claims), “entrained” drug includes drug that is eroded from a mass and dissolved in the vehicle, drug that is eroded from a mass and suspended in the vehicle, and drug that is eroded from a mass and adsorbed/absorbed to nanoparticles or other components of the vehicle. A drug that is removed from a solid drug mass and remains within the vehicle in another chemical form (e.g., a salt that results when a basic solid drug mass is placed into contact with an acidic vehicle) is also included within the scope of the phrase “entrained drug.”

Embodiments of the invention include methods for delivering a therapeutically effective concentration of a drug for which either the acidic or basic form of the drug is water insoluble or sparingly soluble. For a drug with acid-base functional groups, the less water soluble form is likely to be more stable, as a consequence of being less prone to solution-dependent decomposition processes, especially if the drug is stored as a solid, for example in a crystalline state. In addition, as a crystalline or amorphous solid, a drug will occupy the smallest possible space, which also facilitates construction of small delivery devices.

According to at least some embodiments in which the basic form of a solid drug is less soluble than an acidic solid form, solid pellets of the basic form are eluted with an acid at a concentration that is substantially the same as the desired drug concentration. In at least some embodiments in which the acidic form of a drug is less soluble than the basic form, solid pellets of the acidic form are eluted with a base at a concentration that is substantially the same as the desired drug concentration. According to at least some additional embodiments, an aqueous solution comprising one or more components having an amphipathic molecule which can solubilize a water-insoluble drug can be used to erode a solid drug pellet to effect delivery of a therapeutically effective amount of the drug.

An advantage of using solid drug in an implanted device is, in at least some embodiments, the ability to store drug in the device using a smaller volume than might be required if a premixed (or other liquid) form of the drug were used. In some cases, this smaller volume enables implantation of a device containing enough drug to provide (when combined with an appropriate vehicle source) substantially continuous long term therapy. This long term therapy can be over a period of days, weeks, or months. In some cases, long term therapy may extend over several years. One example of a basic crystalline or solid amorphous drug suitable for use in methods according to some embodiments is gacyclidine. For example, it is estimated that 18 mg of solid gacyclidine eroded with an appropriate vehicle will deliver 100 μM drug over 4 years at a flow rate of 20 microliters per hour or less. The hydrochloride salt of gacyclidine, its acidic form, is highly water soluble. However, the acidic form of gacyclidine is also unstable at body temperature. By contrast, the basic form of gacyclidine is sparingly soluble in water and is much more stable than its acidic form in the presence of water. Dissolution of the basic form of gacyclidine in water requires the presence of an acid (e.g., hydrochloric acid or lactic acid) to convert the basic form to the water-soluble acidic form. The concentration of gacyclidine in solution will therefore depend on the amount of acid available to convert the basic form to the acid form. This ability of an appropriate vehicle to change the amount of drug dissolved and delivered offers substantial flexibility in changing the concentration of delivered drug, without requiring the changing of a device holding the solid drug, and without loading a different concentration of a therapeutic solution into a liquid reservoir.

Sterile pellets of gacyclidine base can be prepared by mixing sterile solutions of gacyclidine hydrochloride salt with sterile solutions of sodium hydroxide. Solutions of gacyclidine hydrochloride and sodium hydroxide can be sterilized by passage through a sterilizing filter, such as, but not restricted to, a 0.22 μm polyether sulfone, polytetrafluoroethylene, or polyvinylidene difluoride membrane filter. Polyether sulfone membrane filters have low affinity for gacyclidine solutions at room temperature, pH 5.5 and 25° C.; as such these membranes are compatible with sterile filtration of gacyclidine hydrochloride solutions. After mixing, the solutions are centrifuged to collect the liquid form of drug base into a single mass, which solidifies or crystallizes over time to a single mass of solid drug base. A sterile tube, which forms a mass of the desired shape, can be used in the centrifugation process to prepare sterile pellets of uniform size and shape.

Additional embodiments include methods applicable to delivery of other drugs which are water (or other vehicle) soluble in one of an acid or base form and sparingly soluble in the other of the acid or base form. A solid comprised of the less water soluble drug form is eluted or eroded with a compatible vehicle (e.g., Ringer's solution, Ringer's lactate, saline, physiological saline, artificial vitreous humor and/or any other vehicle compatible with injection into the anterior chamber and/or posterior segment of the eye or into other ocular tissue) comprising, as appropriate, either an acid or a base. If the less water-soluble drug form is a basic form, then the vehicle can contain a pharmaceutically acceptable acid, such as hydrochloric acid, monobasic sodium phosphate (e.g., monosodium phosphate), lactic acid, phosphoric acid, citric acid, a sodium salt of citric acid, or lactic acid. If the less water-soluble drug form is an acidic form, then the vehicle can contain a pharmaceutically acceptable base, such as sodium hydroxide, sodium bicarbonate, or choline hydroxide.

Some embodiments can employ solid drug pellets. Those pellets can be crystalline masses or solid amorphous masses. Examples of manufacturing drug pellets are included herein as Examples 1 and 3. A solid drug could also include a combination of crystalline and amorphous masses. The drug can be melt molded into any desired shape or can be pressed into pellets using pressure (with or without binder). Crystalline drug (if available) may be more desirable than amorphous solid drug forms in some cases, as crystalline substances typically are more stable. Crystal lattice energy may also help stabilize the drug. However, the invention is not limited to crystalline drug forms or the use thereof.

The invention is similarly not limited to drugs (or to methods or devices employing drugs) with acid-base functionalities. Embodiments also include dissolution (or removal from a mass by other mechanism) of any drug which is sparingly soluble in water by eluting the drug with a pharmaceutically acceptable vehicle comprising one or more components having an amphipathic molecule, such as monopalmitoyl glycerol or polysorbate 80 (e.g., TWEEN 80®). Other suitable amphipathic molecule components include (but are not limited to) an acyl glycerol, a poly-oxyethylene ester of 12-hydroxysteric acid (e.g., SOLUTOL® HS15), beta-cyclodextrin (e.g., CAPTISOL®), a bile acid such as taurocholic acid, tauroursodeoxycholic acid, cholic acid or ursodeoxycholic acid, a naturally occurring anionic surfactant such as galactocerebroside sulfate, a naturally occurring neutral surfactant such as lactosylceramide or a naturally occurring zwitterionic surfactant such as sphingomyelin, phosphatidyl choline or palmitoyl carnitine. Dissolution (or other removal) can also be accomplished by use of physiological fluid vehicles, such as interstitial fluid or natural (or simulated) tear fluid. Physiological fluid vehicles contain amphipathic molecules, such as proteins and lipids, which are capable of effecting dissolution of a water-insoluble drug. Dissolution can also be carried out without the use of an amphipathic molecule where an acceptable concentration of drug is obtained.

One example of a drug that does not have acid-base functionalities is triamcinolone acetonide. Triamcinolone acetonide is commercially available as a crystalline solid with very low water solubility. If solid pellets of triamcinolone acetonide are exposed to a continuous stream of a vehicle, such as Ringer's solution, the expected concentration of extracted triamcinolone acetonide in solution should be 40 μM or less. A higher concentration of triamcinolone acetonide can be solubilized by including an amphipathic molecule in the vehicle. Such a pharmaceutically acceptable amphipathic molecule would be polysorbate 80 (e.g., TWEEN 80®). The concentration of triamcinolone acetonide solubilized can be increased above its water solubility, 40 μM, by adding the required amount of amphipathic molecule to the vehicle that will support the desired drug concentration. The invention is not limited to methods implemented through use of triamcinolone acetonide, Ringer's solution or polysorbate 80. Any sparingly soluble drug, pharmaceutically acceptable vehicle and pharmaceutically acceptable amphipathic molecule can be used.

Still other embodiments employ nanoparticles. Nanoparticles can maintain a drug in a mobile phase capable of passing through an antibacterial filter. Some embodiments would use, in place of or in combination with an amphipathic drug carrier, a suspension of particles (e.g., nanoparticles) that would have affinity for a drug (e.g., that would adsorb/absorb a drug) and act as carriers. Yet other embodiments include use of pure drug nanoparticles. Embodiments also include combinations of both pure drug nanoparticles and drug adsorbed/absorbed to carrier nanoparticles. Particles according to at least some embodiments would be small enough to pass through an antibacterial filter of 0.22 microns or less. Removal of a drug from a mass thereof using a vehicle having suspended carrier nanoparticles would be advantageous to both drug stability and delivery. Removal of solid drug from a mass of drug nanoparticles would have similar benefits.

In at least some embodiments a vehicle includes a suspension of small carrier particles (100 nm to 0.1 mm in size) or carrier nanoparticles (10 nm to 100 nm in size) having an affinity for the drug(s) to be delivered. Examples of materials from which the carrier particles or nanoparticles could be formed include (but are not limited to) polylactic acid, polyglycolic acid, a co-polymer of lactic acid and glycolic acid, polypropylene, polyethylene and polystyrene. Additional examples of materials from which carrier particles or nanoparticles can be formed include magnetic metals and magnetic metals having a coating to attract a drug (or drugs) of interest. These small carrier particles or nanoparticles will adsorb/absorb or otherwise attract drug that is eroded from a mass of solid drug (which may be stored in a reservoir such as is described herein) by a vehicle in which the carrier particles (or nanoparticles) are suspended.

In some embodiments, a vehicle will be used to erode pure drug nanoparticles from a solid mass composed of such pure drug nanoparticles. Such a solid mass of nanoparticles could be formed by compression and/or by use of a binder.

In some cases, a small amount of acid or amphipathic excipient (e.g., SOLUTOL® HS15, TWEEN 80® or CAPTISOL®) can be employed to facilitate drug elution from a mass of solid drug (or from a mass of solid drug nanoparticles) and transfer of the drug into solution or into a mobile nanoparticle suspension.

In some embodiments, polymeric material used to fabricate carrier nanoparticles is biodegradable (so as to help promote ultimate delivery of drug), commercially available and approved for human use. Polymers of L- and D,L-lactic acid and copolymers of lactic acid and glycolic acid [poly(lactide-co-glycolide)] (available from Lakeshore Biomaterials in Birmingham, Ala.) are examples of polymeric materials that have the potential to meet the desired properties of the polymer for carrier nanoparticles. Nanoparticles small enough to pass through a 0.22 μm antibacterial filter have been fabricated from a 50:50 mix of poly(lactide-co-glycolide) by the solvent displacement method.

Several methods have been employed to fabricate nanoparticles of suitable size. These methods include vaporization methods (e.g., free jet expansion, laser vaporization, spark erosion, electro explosion and chemical vapor deposition), physical methods involving mechanical attrition (e.g., pearlmilling), interfacial deposition following solvent displacement and supercritical CO₂. Additional methods for preparing nanoparticles include solvent displacement of a solubilizing solvent and a solvent in which the nanoparticle is not soluble, vibrational atomization and drying in the atomized state, sonication of two liquid streams, use of micropumps (such as ink jet-like systems delivering nano and micro-sized droplets of drug) and continuous flow mixers.

When preparing nanoparticles by the solvent displacement method, a stirring rate of 500 rpm or greater is normally employed. Slower solvent exchange rates during mixing produce larger particles. Fluctuating pressure gradients are fundamental to producing efficient mixing in fully developed turbulence. Sonication is one method that can provide adequate turbulent mixing. Continuous flow mixers (two or more solvent streams) with and without sonication may provide the necessary turbulence to ensure small particle size if the scale is small enough. The solvent displacement method has the advantage of being relatively simple to implement on a laboratory or industrial scale and has produced nanoparticles able to pass through a 0.22 μm filter. The size of nanoparticles produced by the solvent displacement method is sensitive to the concentration of polymer in the organic solvent, to the rate of mixing and to the surfactant employed in the process. Once isolated, a dried or wet pellet of drug particles or drug-laden polymeric particles can be compressed into a solid mass or mixed with a pharmaceutically acceptable binder and compressed into a mass.

### Part II: Ocular Drug Delivery Devices

Drug-delivery systems according to at least some embodiments include combinations of various implantable components. These components include osmotic pumps, subcutaneous (or transdermal) ports, catheters and terminal components. In some cases, an osmotic pump (and/or a port) and other system components are small enough to permit subcutaneous implantation on the side of a patient's head (or elsewhere on the head), and can be used for delivering drugs to the eye. These components can also be implanted elsewhere on a patient's body, however.

In at least some embodiments, a device employed for removal of drug from a solid drug mass with (and entrainment by) a vehicle can include any chamber capable of holding a less water-soluble form of the drug and permitting a vehicle comprising a dissolving or other removal agent (e.g., acid, base, an amphipathic molecule, a suspension of nanoparticles) to flow past the solid drug. The size of the chamber, rate of vehicle flow and concentration of acid, base, amphipathic molecule or nanoparticles used are determined by the intended application of the drug delivery device and dissolution characteristics (or erosion or other physical characteristics) of the drug substance and/or drug mass, as well as by any required vehicle reservoir and/or pumping system. Determination of the parameters for such a device is within the ability of one skilled in the art, once such a person is provided with the information included herein.

Fluid flow to effect drug dissolution (or removal by other mechanism) can be accomplished by any pump with fluid flow parameters that match the desired application. Such pumps include, but are not limited to, an implantable MEMS pump, an implantable osmotic pump, an implantable peristaltic pump, an implantable piston pump, an implantable piezo-electric pump, etc. Selection of an appropriate pump is similarly within the ability of one skilled in the art, once such a person is provided with the information included herein. In some embodiments, a pump can be fully implanted within a human (or animal) body. In other embodiments, a pump may be external to the body and delivering vehicle through a subcutaneous port or other connection to a reservoir holding solid drug.

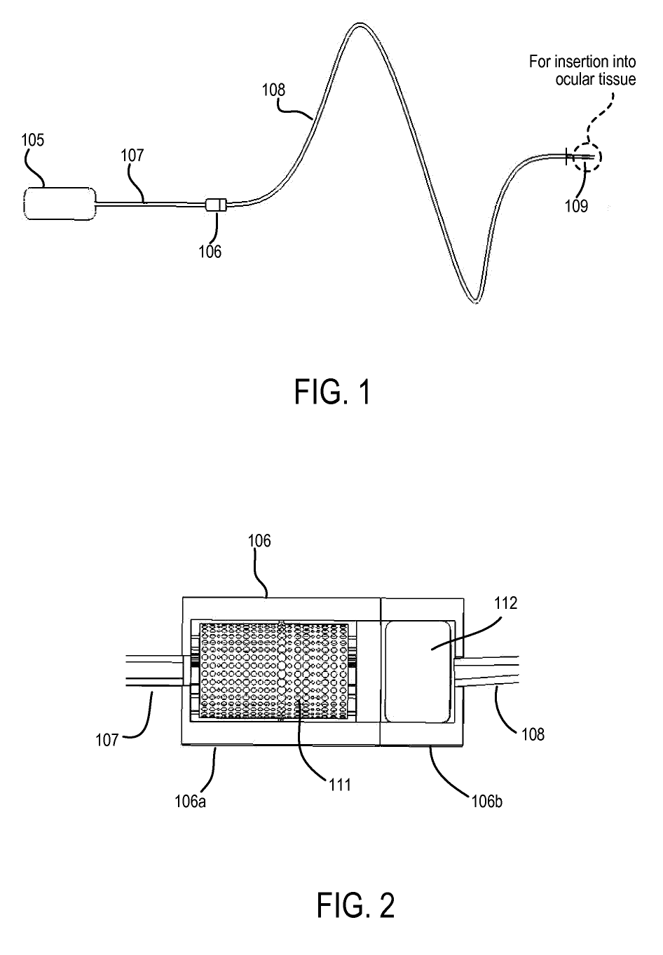

FIG. 1 is a drawing of a drug delivery system, according to at least some embodiments, that can be used to deliver drug from a solid drug mass. The system of FIG. 1 includes an implantable osmotic pump 105 and a drug/filter housing 106. As explained below, housing 106 includes an internal cavity, an inlet and an outlet. A lumen of first catheter 107 connects an outlet of osmotic pump 105 and an inlet of drug/filter housing 106. A “catheter” is a tube or other slender body having one or more internal lumens through which a fluid may flow. A lumen of second catheter 108 connects an outlet of drug/filter housing 106 to a terminal component 109. As can be appreciated, a fluid path is formed by pump 105, the lumen of catheter 107, the internal cavity of housing 106, the lumen of catheter 108, and terminal component 109.

Osmotic pump 105 is of a type known in the art. Such pumps (e.g., pumps sold under the trade names DUROS® and CHRONOGESIC® by Durect Corp. of Cupertino Calif.) are known for use in other applications, and are described in, e.g., U.S. Pat. No. 4,034,756. In general, an implanted osmotic pump incorporates osmotic pressure differences to drive a drug at a predefined flow rate related to the aqueous permeability of a membrane in the pump. This mechanism typically uses an osmopolymer, salt, or other material with high osmolality to imbibe liquid from the surrounding tissue environment and expand a compartment volume. This volume increase moves a piston or compresses a flexible reservoir, resulting in expulsion of a liquid from the pump. The piston (or a moveable seal) separates the osmopolymer from a reservoir containing the liquid to be expelled. The pump housing may consist of a semi-permeable body which allows water or appropriate liquid to reach the osmopolymer. The rate of delivery of the pump is determined by the permeability of the pump's outer membrane.

Conventional osmotic pumps hold a liquid formulated drug in the liquid reservoir; such pumps can be used to deliver such a liquid drug formulation to an eye or other ocular tissue in some embodiments. Osmotic pump 105 in FIG. 1, however, contains a drug vehicle. The vehicle is expelled from pump 105 for entrainment of a drug from a solid drug mass inside of drug/filter housing 106. In other embodiments, pump 105 may expel a liquid that contains a drug, but which is also used as a vehicle to carry an additional drug from drug/filter housing 106.

Osmotic mini-pumps can deliver small amounts of liquid continuously for long periods of time. However, it can be difficult to refill an internal fluid reservoir of a conventional osmotic pump. Accordingly, the embodiment of FIG. 1 includes a fitting (not shown in FIG. 1) that allows convenient removal and replacement of osmotic pump 105 in a brief surgical procedure. Controlling the flow rate of an osmotic pump can also be difficult. Variations on the embodiment of FIG. 1 include a controllable valve connected to the pump which isolates the semi-permeable membrane (within the pump) from low osmolality environmental fluids. This prevents entry of the fluid into the pump compartment to drive the fluid delivery piston. The control valve may be a piezoelectric element which deforms when an electrical field is applied across it. Such a valve may be connected and controlled by an internal electronics package or by an internal control module which receives signals through RF transmission (e.g., from an external signal system worn by the patient outside the body). In still other embodiments, a small magnetically activated switch is built into the electronics for the valve. The valve is opened or closed by placing a magnet of sufficient strength over the portion of the patient's body where the control electronics have been implanted. Similar magnetically activated switches are found in implanted devices such as pacemakers and implanted cardiac defibrillators. Even when such control valves are employed, however, an osmotic pump may not function in an instant-on/instant-off manner. For example, there may be a delay between the time a control valve is closed and the time that the pump delivery tapers off; during this delay the pump is reaching osmotic equilibrium. In yet other embodiments, this can be addressed by placing a control valve or a diverter valve on the pump outlet catheter 107. In still other embodiments, a pressure release valve could be included to drain away osmotic pressure in emergency situations requiring immediate pump shutdown.

FIG. 2 is a cross-sectional view of drug/filter housing 106 from FIG. 1. Housing 106 serves as a capsule to hold one or more solid drugs and an antibacterial filter. In some embodiments in which an implanted osmotic pump is used to deliver a liquid drug formulation, housing 106 may only contain an anti-bacterial filter. Housing 106 is formed from titanium or other material which is both biocompatible and compatible with drugs to be dispensed. A proximal (or “upstream”) end of housing 106 holds a porous cage 111 which may be permanently attached to the housing, or which may be removable. Cage 111, which is also formed from titanium or other bio- and drug-compatible material(s), holds one or more masses of one or more solid drugs. The drug(s) may be monolithic, in the form of a powder, in the form of pellets, or in some other solid configuration. Multiple holes on cage 111 allow fluid from pump 105 to mix with and carry away a portion of that solid drug in dissolved (or other entrained) form. A distal (or “downstream”) end of housing 106 contains a three-dimensional antibacterial filter 112. As described in more detail below, an “antibacterial filter” is a filter having a pore size that is small enough to allow a drug-carrying fluid to pass, but which obstructs passage of bacteria or other undesirable elements. Housing 106 is a two piece assembly (pieces 106a and 106b), thereby allowing housing 106 to be taken apart and reassembled to replace cage 111 (e.g., to change drug or when the drug is depleted) and/or filter 112 (e.g., if the filter becomes clogged). Pieces 106a and 106b can be attachable to one another via threaded connection or by other type of mechanical mechanism (e.g., interlocking tabs and slots). Catheter 107 is attached to an inlet in piece 106a; catheter 108 is attached to an outlet in piece 106b. Catheters 107 and 108 may be attached with epoxy or other adhesive. In other embodiments, barbed connectors may be employed. Clips and/or other locking mechanisms could also be used to retain catheters 107 and 108 to housing 106.

In at least some embodiments, osmotic pump 105 and drug/filter housing 106 are sized for implantation in specially prepared pockets in a patient's skull. Catheters 107 and 108 may be placed within grooves also prepared on the patient's skull.

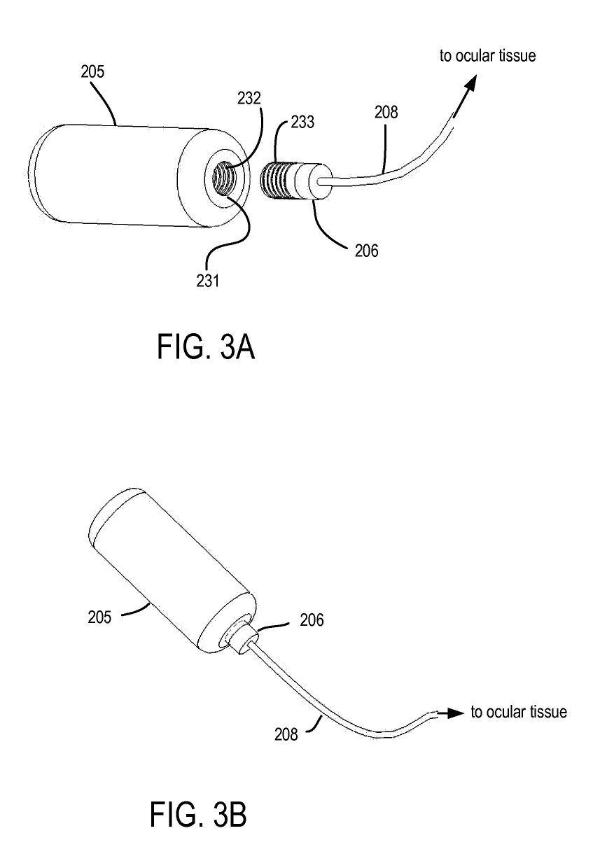

FIGS. 3A and 3B show a drug delivery system according to another embodiment. Osmotic pump 205 is similar to osmotic pump 105 of FIG. 1, except that outlet 231 of pump 205 is somewhat enlarged and has internal threads 232. Drug/filter housing 206 is similar to housing 106 of FIGS. 1 and 2. However, housing 206 has external threads 233 corresponding to internal threads 232 on outlet 231 of pump 205. As shown in FIG. 3B, this facilitates a direct attachment between pump 205 and housing 206, thereby avoiding the need for one of the catheters (i.e., catheter 107) shown in FIG. 1. An inlet to housing 206 (similar to the inlet of housing 106 connected to catheter 107 in FIG. 2) is placed into fluid communication with the outlet of pump 205. Fluid from an outlet of housing 206 flows to an ocular tissue through catheter 208. The dimensions of the housing 206 will depend on the drug(s) being delivered and the surface area required to provide a desired concentration of the drug(s).

The configuration of FIGS. 3A-3B allows periodic removal of housing 206 from pump 205 for replacement of drug and/or a filter within housing 206. In variations on the embodiment of FIGS. 3A-3B, other types of connection mechanisms (e.g., locking tab and groove) between pump 205 and housing 206 are employed. In still other variations, housing 206 is permanently attached (e.g., with adhesive) to pump 205.

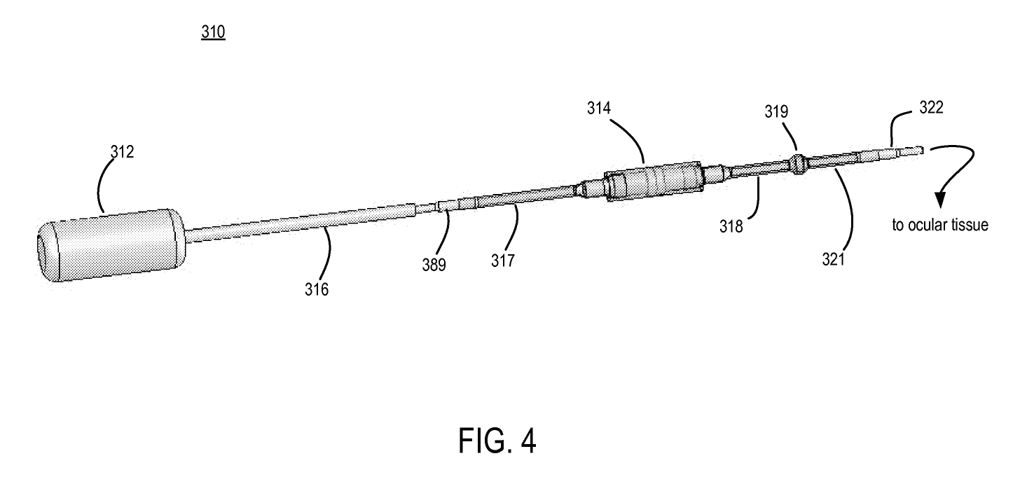

Another embodiment of an ophthalmic drug delivery device is shown in FIG. 4. In the embodiment of FIG. 4, device 310 includes an osmotic pump 312 coupled to a sleeved drug reservoir 314 via catheters 316 and 317. A three-dimensional (3-D) antibacterial filter 319 is coupled to drug reservoir 314 via a catheter 318. Another catheter 321 and connector 322 connects 3-D filter 319 via an additional catheter (not shown) to a terminal component (also not shown) positioned for delivery of a drug-laden solution into the target ocular tissue. The terminal component may be, e.g., a needle or an open end of a catheter. Prior to implantation, the osmotic pump is filled with a solution that will entrain the solid drug.

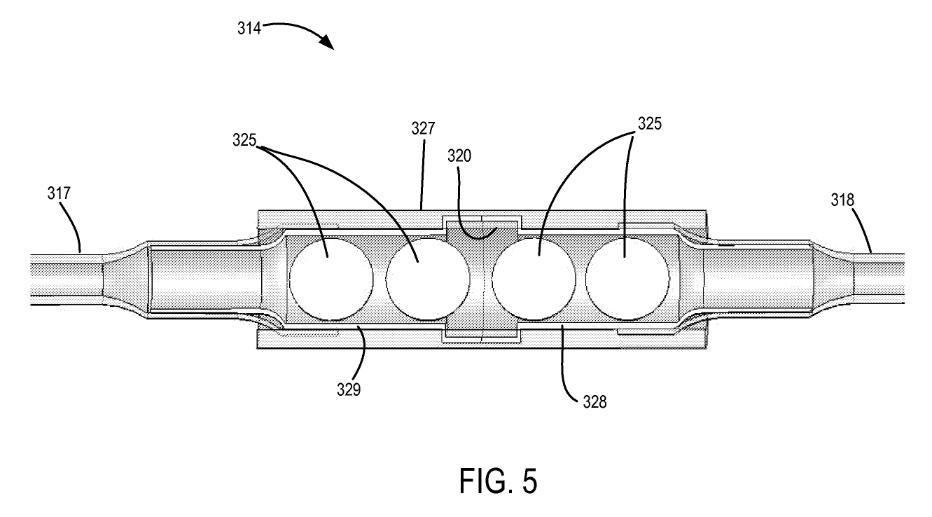

A solid drug reservoir is designed to provide a cavity for fluid to flow around and erode one or more masses of solid drug (e.g., solid drug pellets). FIG. 5 is a cross-sectional view of sleeved drug reservoir 314 of FIG. 4, which is but one example of a drug reservoir according to at least some embodiments. Drug reservoir 314 includes two hollow metal tubes 328 and 329 (made from a drug compatible material) forming a chamber 320 into which one or more solid drug pellets 325 are loaded. A sleeve 327 (made from silicone or other appropriate material) is rolled over tubes 328 and 329 to form a liquid tight seal. Tapered ends of tubes 328 and 329 fit into ends of catheters 318 and 317, respectively. Drug reservoir 314 of FIG. 5 is shaped to contain the drug pellets within chamber 320 and prevent solid pieces from moving out of chamber 320. Drug reservoir 314 may also be pulled apart and reattached to thereby allow loading of one or more solid drug pellets.

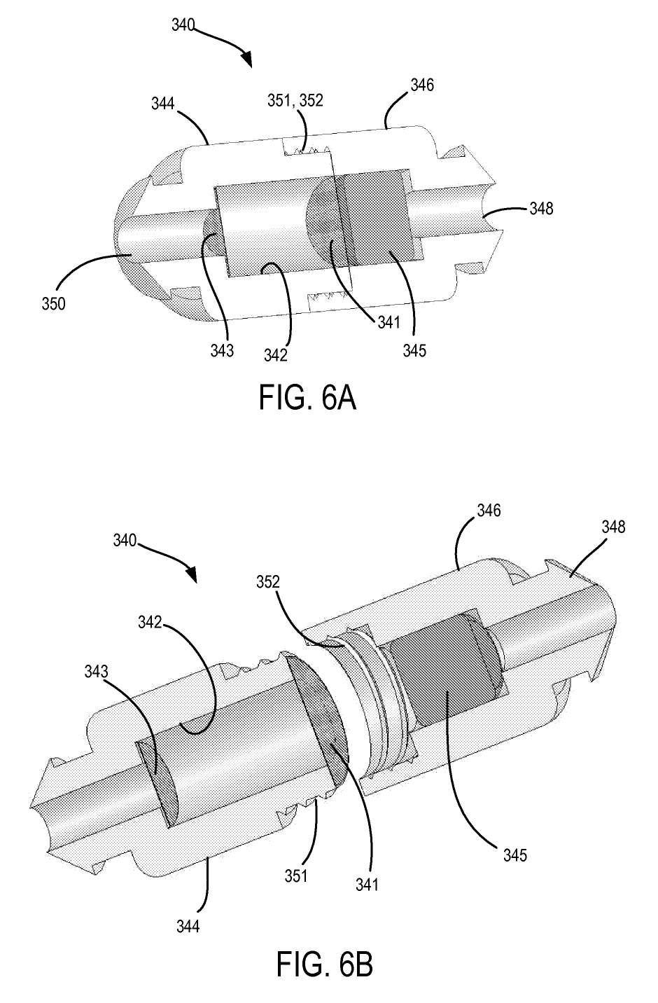

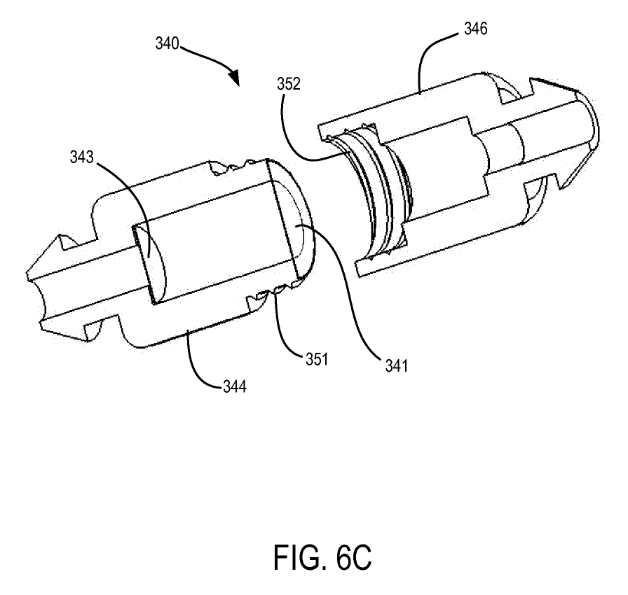

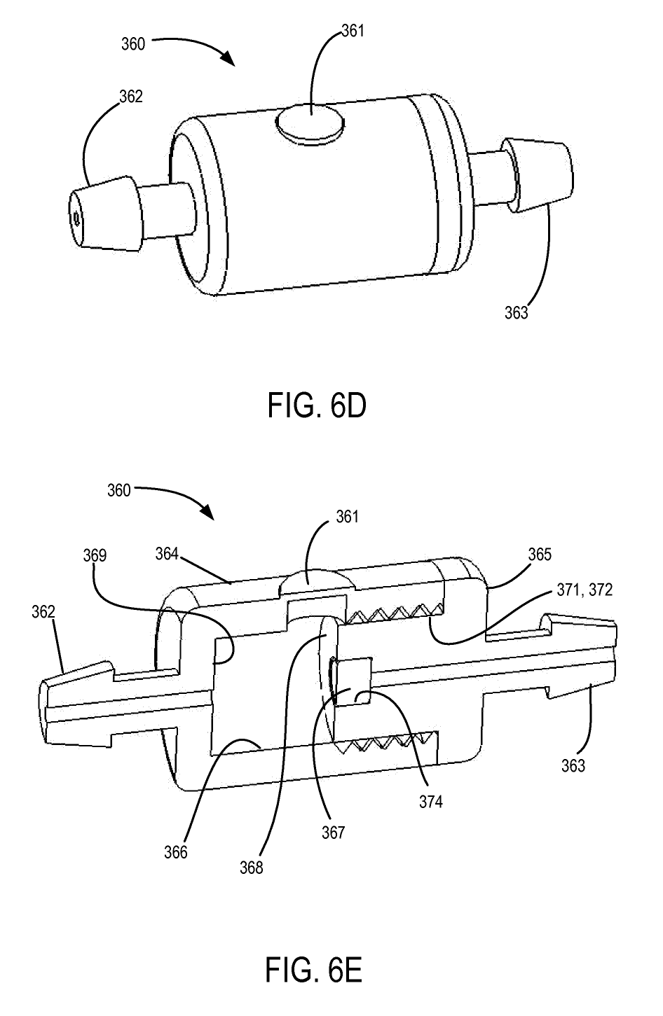

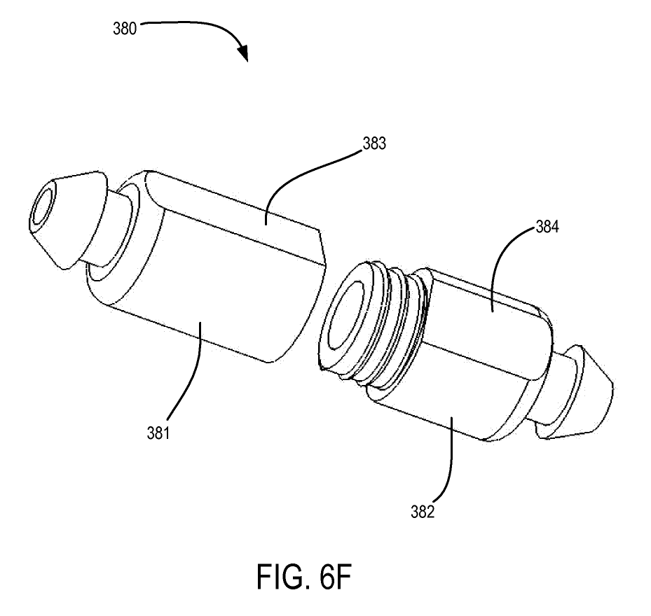

In some embodiments, circular screens are placed inside a drug chamber to further prevent migration of drug pellets. In some cases, at least one of the screens may be removable to allow for replenishment of drug. FIGS. 6A and 6B are cross-sectional views of a drug reservoir 340 according to another embodiment, and that includes such screens. As seen in FIGS. 6A and 6B, drug reservoir 340 includes housings 344 and 346 that mate together (with threads 351 and 352) to form a fluid-tight connection. Solid drug can be placed inside chamber 342 within housing 344, with housing 344 including a stationary meshed screen 343 on the side of tubing connection inlet 350 and a removable meshed screen 341 at the edge of housing 344. As seen in FIG. 6A, screen 341 is directly before 3-D antibacterial filter 345, which rests within housing 346. Screens 341 and 343 are porous and may be woven wire cloth made of titanium, stainless steel, or other biocompatible, drug compatible metals (e.g., gold, platinum) and/or polymers (e.g., fluoropolymers). In other embodiments, the screens may be made of porous metal, such as titanium or stainless steel. Meshed screens 341 and 343 prevent drug pellets from going into the housing 346, antibacterial filter 345 or tubing (not shown) that may be connected to inlet connection 350 or outlet connection 348. In FIG. 6A drug reservoir 340 is shown with housing halves 344 and 346 threaded together. FIG. 6B shows housings 344 and 346 separated, but with removable screen 341, stationary screen 343 and antibacterial filter 345 in place. As seen in FIG. 6B, removable screen 341 covers the outer circular surface of the end of housing 344. Stationary screen 343 only covers the inner circular surface of space 342. Screens can be of any shape to fit the shape of the drug chamber. Screens are not required, however, and may be omitted in certain embodiments.

An antibacterial filter is similarly not required. For example, FIG. 6C is a cross-sectional view of drug reservoir 340 without antibacterial filter 345. At least some embodiments may also include features which permit air bubbles to bleed off during filling of the system. This can help to prevent vapor lock in cases where a fluid delivery system (e.g., an osmotic pump or an external pump connected through a subcutaneous port) does not generate sufficient pressure to overcome surface tension holding liquid within capillary-like structures of a wet porous filter (such as 3-D filter 345 of FIGS. 6A and 6B). In some embodiments, a set screw or plug may be incorporated into the side of a drug chamber housing on the upstream (i.e., higher pressure) side of the filter. The set screw or plug may be removed during priming and reattached for use once all air bubbles have been bled from the system. In still other embodiments, a vent valve may include an upstream semi-permeable membrane allowing for venting of gases. In yet other embodiments, the set screw or plug may be non-removable, but may include a portion which is gas-permeable but not liquid-permeable so as to allow degassing.

FIG. 6D shows a drug reservoir 360 according to at least one embodiment, and which includes vent valve 361 having a semi-permeable membrane allowing for venting of gases. Tubing connector barb 362 is on the upstream side of reservoir 360, and tubing connector 363 is on the downstream side. FIG. 6E is a cross sectional view of drug reservoir 360. Drug reservoir 360 includes housings 364 and 365 which join to form a fluid-tight connection with threads 371, 372. A cavity 366 holds one or more solid drug pellets or other masses. Although not shown, screens similar to screens 343 and 341 in FIGS. 6A and 6B can be placed (in either a stationary or removable configuration) over face 369 on the upstream side of space 366 and over face 368 on the downstream side of space 366. In the embodiment of FIG. 6E, a 3-D antibacterial filter 367 fits within a space 374 formed in housing 365.

Housings 344 and 346 of drug reservoir 340, housings 364 and 365 of drug reservoir 360, and housings of drug reservoirs in other embodiments can be made of a drug-compatible, corrosion-resistant material such as titanium, stainless steel, platinum, gold, a biocompatible coated metal, a chemically inert polymer such as PTFE (polytetrafluoroethylene), FEP (tetrafluoroethylene-hexafluoropropylene copolymer), PFA (perfluoroalkoxyethylene), other fluoropolymers, or a fluoropolymer-coated metal. During low flow rates at body temperature, drug may tend to adsorb to the walls of the chamber, causing lower than expected concentrations of drug to be delivered to the patient. Fluoropolymers are the best known materials for resisting adsorption. Other fluoropolymers include, but are not limited to, ECTFE (ethylene-chlorotrifluoroethylene copolymer), ETFE (ethylene-tetrafluoroethylene copolymer), MFA (tetrafluoroethylene perfluoro(methylvinyl ether) copolymer), PCTFE (polychloro tri-fluoro ethylene) and PVDF (polyvinylidene difluoride).

As indicated above, drug reservoirs in various embodiments may be opened and closed to allow for replenishment of solid drug. The reservoir components may be threaded (as shown in FIGS. 6A-6C and 6E) or may consist of a locking tab and groove. In still other embodiments an external clamp may be used. In yet other embodiments, reservoir housings may be joined by a snap-fit. As also indicated above, reservoir 314 (FIG. 5) includes two metal tubes 328 and 329 held together by a surrounding sleeve 327. Surrounding sleeve 327 may be made of a flexible polymer such as silicone rubber. In some embodiments, a biocompatible gasket can be placed between mating portions of a drug reservoir (e.g., between tubes 328 and 329 of FIG. 5, between housings 344 and 346 of FIGS. 6A-6C, between housings 364 and 365 of FIG. 6E) to prevent leaks. In still other embodiments, external portions of a drug reservoir housing may include flats or other regions to facilitate easier tightening. FIG. 6F shows an embodiment of a drug reservoir 380 having mating housings 381 and 382. A flat 383 is formed on one side of housing 381. A second flat (not shown) can be formed on an opposite side of housing 381. Similarly, housing 382 includes a flat 384 formed on one side, and can also include an additional flat (also not shown) on an opposite side.

A drug cage similar to drug cage 111 (FIG. 2) can be used with any of the drug filter housings shown in FIGS. 5-6F, as well as with other housings described below.

In at least some embodiments, catheter tubing on the upstream side of a drug reservoir (e.g., tubing for catheter 316 on the pump side of device 310 in FIG. 4) is a vehicle- and biocompatible, flexible polymer such as silicone, polyurethane, or fluoropolymer including PTFE, FEP, and PFA and the catheter tubing on the downstream side of the drug reservoir is a biocompatible, drug compatible, flexible polymer such as PTFE, FEP and other fluoropolymers.

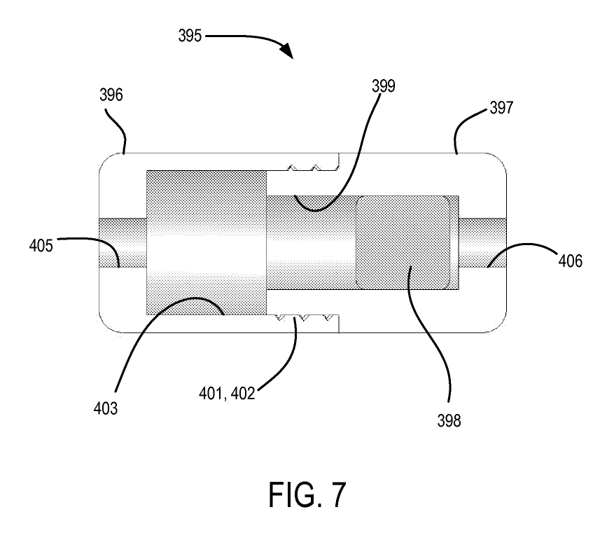

In some embodiments, the solid drug reservoir and a 3-D antibacterial filter are in fluid communication via catheter connection. This is seen generally in FIG. 4, which also shows metal tubing connectors 322 and 389 that can be used to connect to upstream or downstream components. In other embodiments, and as described above, a single housing may contain solid drug (alone or in a cage) as well as a three-dimensional antibacterial filter. Such a housing may also be opened and closed to allow for replenishment of solid drug. FIG. 7 is a cross-sectional view of a drug reservoir 395 according to another embodiment. Drug reservoir 395 includes housings 396 and 397 joined by mating threads 401, 402. A cavity 403 inside housing 396 holds solid drug (not shown). Screens similar to screens 341 and 343 of FIGS. 6A and 6B may also be included. Optionally, a 3-D antibacterial filter 398 is located in a space 399. Instead of the barbed fittings shown in FIGS. 6A-6F, drug reservoir 395 includes an upstream inlet hole 405 and a downstream outlet hole 406.

In at least some embodiments, a housing for a drug and filter is made from titanium, gold, platinum or stainless steel and is small enough to be implanted into a human body. The inner diameter is sized so that a 3-D antibacterial filter can be bonded to the inside of the housing. Examples of possible filter sizes (in various embodiments) include but are not limited to 0.22 micron maximum pore size 3-D filters with a physical outer diameter of 0.03 to 0.25″. In still other embodiments the physical outer diameter is between 0.1″ and 0.3″.

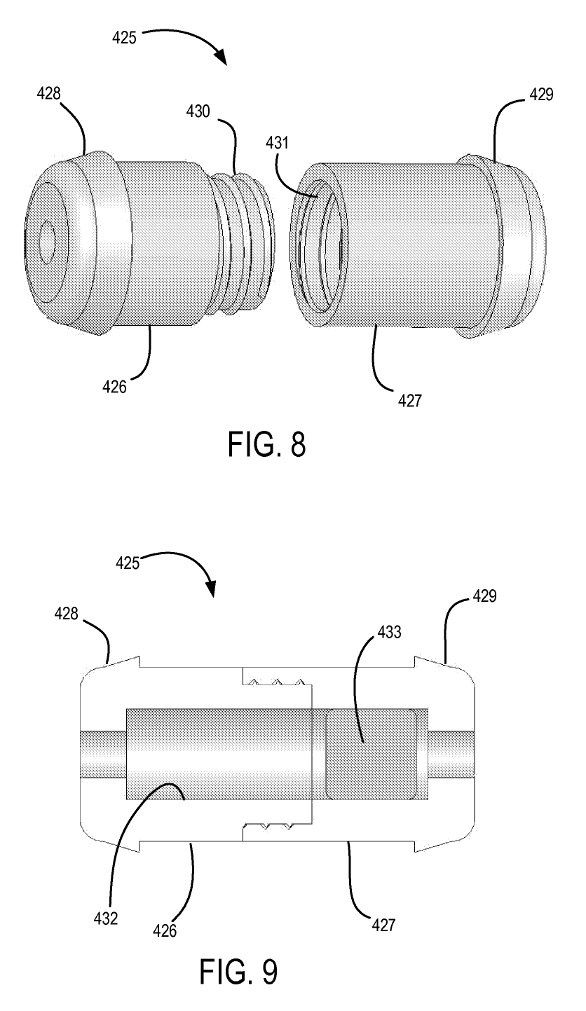

FIG. 8 is a perspective view of two separated housings 426 and 427 of a drug reservoir 425 according to at least one embodiment. FIG. 9 is a cross-sectional view of drug reservoir 425, with housings 426 and 427 joined (via threads 430 and 431). The entire outer ends of housings 426 and 427 have barbs 428 and 429 (respectively) formed thereon. Also seen in FIG. 9 are a space 432 for holding solid drug and an optional 3-D antibacterial filter 433.

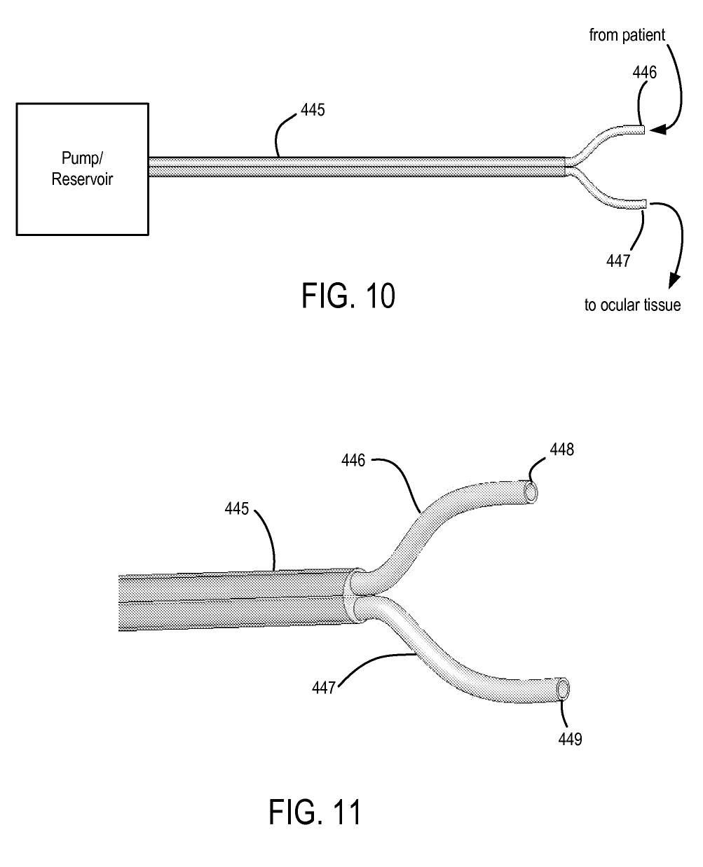

FIG. 10 shows an additional embodiment in which a dual lumen tube 445 extends from a pump and/or reservoir containing solid drug. Dual-lumen tube 445 separates into two separate lines. Tube 446 is attached to one lumen and receives inflowing physiological fluid from a patient. Tube 447 is attached to another lumen and delivers therapeutic fluid to the ocular tissue of a patient. Interstitial fluid received in line 446 flows past solid drug pellets in the reservoir and slowly removes (e.g., by dissolution) drug from those pellets. The resulting solution of drug and physiological fluid is then delivered to the target ocular tissue through tube 447. FIG. 11 is an enlarged view of the distal ends 448 and 449 of tubes 446 and 447, and further illustrates the two lumens for recirculating fluid flow. In other embodiments, two completely separate tubes (i.e., two tubes that do not emerge from a dual lumen tube) may be used. Such an embodiment could be useful in cases where physiological fluid is withdrawn from a region that is more distant from the region in which therapeutic fluid is to be delivered. In certain embodiments, some or all of fluid received from an eye through tube 446 is not recirculated. This could take place so as to, e.g., reduce excess intra-ocular pressure caused by glaucoma.

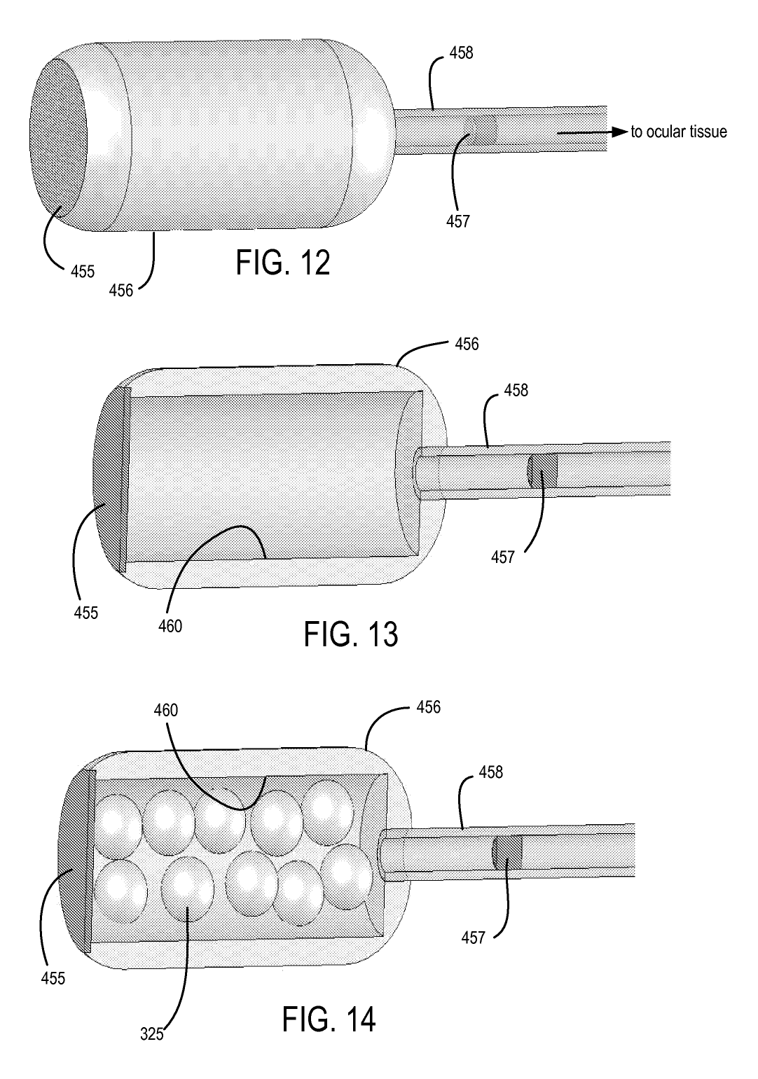

FIG. 12 is a perspective view showing an embodiment of a system which does not require a pump to generate flow. A semi-permeable membrane 455 allows an interstitial fluid vehicle to pass into a chamber of a reservoir 456 containing solid drug. As drug within the chamber dissolves (or is otherwise removed from the solid drug mass and entrained in the interstitial fluid vehicle), the concentration difference across the membrane causes fluid to flow from low concentration to higher concentration. Osmotic pressure forces fluid past membrane 455, into the drug chamber, through the outlet, and past an optional 3-D antibacterial filter 457 in a catheter 458 (shown as a clear catheter for purposes of illustration) to the target ocular delivery site. Semi-permeable membrane 455 has a pore size cutoff sufficient to let interstitial fluid through but not let the entrained solid drug diffuse out. Antibacterial filter 457 has pores sufficient to retain bacteria but to let dissolved (or otherwise entrained) drug pass through. An electric field may also be applied to membrane 455 resulting in diffusion by electro-osmosis. FIG. 13 is a fully cross-sectional view of the embodiment of FIG. 12, and shows in more detail a cavity 460 for holding a solid drug. FIG. 14 shows the embodiment of FIGS. 12 and 13 containing solid drug pellets 325 in cavity 460. Appropriate check valves (not shown) can be included within cavity 460 or elsewhere in the fluid path so as to prevent backflow.

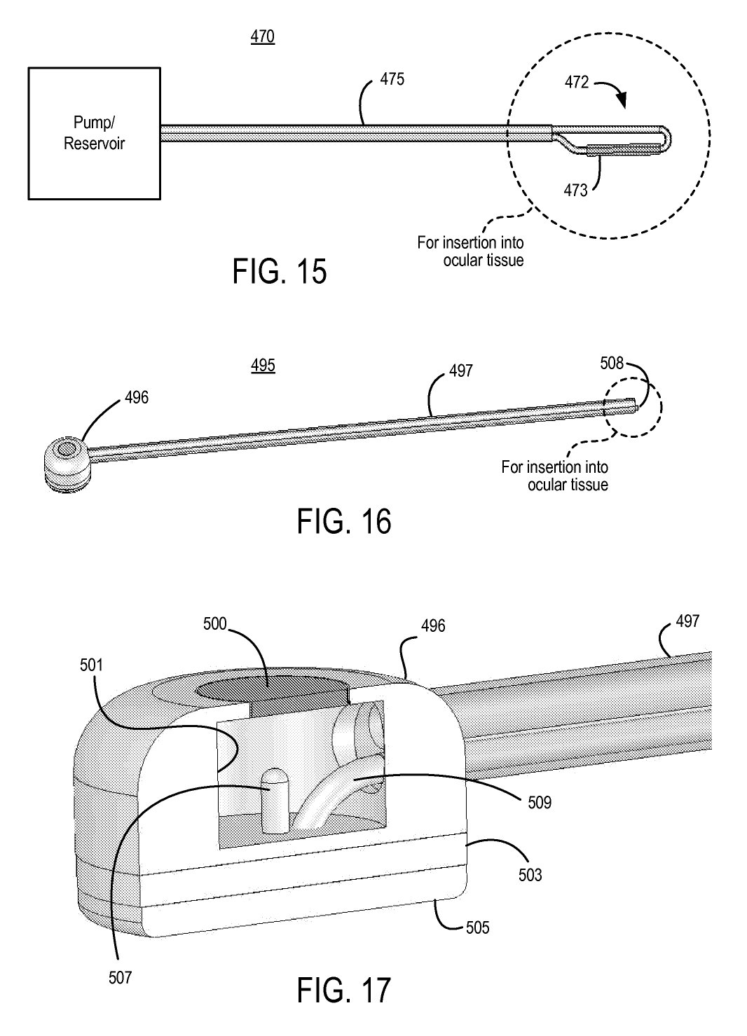

FIG. 15 shows an embodiment of a system 470 where fluid is circulated unidirectionally from a pump/reservoir (via one lumen of dual-lumen tubing 475) through a loop 472 containing a semi-permeable hollow fiber 473 and returned through a second lumen of tubing 475. Hollow fiber loop 473 is a terminal component which can be positioned at a target ocular delivery area. The pump circulates vehicle past solid drug located in the reservoir, and the resulting drug-loaded vehicle diffuses through the walls of hollow fiber 473 into the target ocular tissue. In other embodiments, a delivery system similar to that of FIG. 15 contains a drug permeable hollow fiber which will release drug into the external environment by passive diffusion, but without actually delivering a volume of liquid.

Still other embodiments include sensors (e.g., a pressure sensor for glaucoma or a drug sensor) with attached battery and power electronics (power supply, recharging circuitry, etc.) and communication electronics to receive and send information. In these embodiments, the electronics could be bundled with the reservoir section of the device and the sensors could be combined with a wire following the surface of the catheter or contained within one of the lumens of a multi-lumen tubing and exiting within a target ocular tissue.

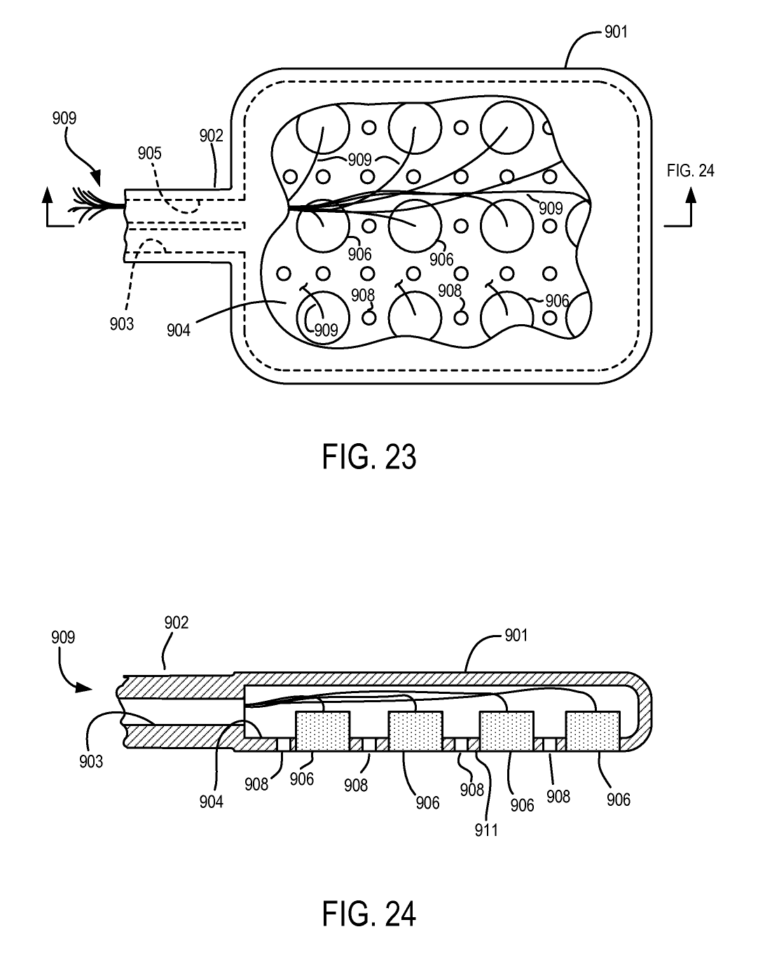

At least some embodiments include electrophoresis-stimulated delivery of charged drug ions or other particles of drug. For charged drugs, applying an electric field on a fluid containing the drug (or containing nanoparticles that have adsorbed/absorbed drug) can induce the migration of the drug faster than normal diffusion. In the case of gacyclidine, a negative charge on a device exit (e.g., at the end of a catheter) or just outside of a device exit can be used to accelerate the drug delivery to the eye without the need for a pump. A same or similar charge of opposite polarity (e.g., a positive charge in the case of gacyclidine) could similarly be applied to a drug containing compartment (e.g., a chamber in which solid drug is held), thereby enabling drug delivery out of the device without the need for a pump. The electrophoresis environment would induce an electro-osmotic flow to the natural low resistance outlet within the target ocular tissue. The rate of migration of drug to the catheter tip (or the concentration of drug) could be modulated by field strength of the electric charge and other parameters modulated by an appropriate electronics package, battery, recharging assembly, on/off switch, communication circuitry and other electronics. If a drug having an opposite charge is used, then the electronic circuitry would reverse the charges on the electrodes. Electrophoresis-stimulated drug delivery embodiments would be very low power devices in order to promote patient safety, and because small amounts of drug are being delivered. A charged device in an ocular tissue may provide additional benefits to suppress neural degeneration of the optic nerve, e.g., in blind patients and in special circumstances to treat patients with light flashes in the eye or a hyperactive sensitivity to light, as well as to other patients who report benefit from electrical stimulation. In some embodiments (and as described below in connection with FIGS. 23 and 24), a catheter includes an electrode that is only used for delivery of electrical stimulation (pulsed or otherwise) to the eye. In still other embodiments, a catheter includes an electrode that is alternatively (or additionally) used to sense intra-ocular pressure, electrical potential or some other physical characteristic in the eye. Methods and electronics for such stimulation and/or sensing are known in the art (although not in combination with the drug delivery devices described herein). Inclusion of appropriate stimulation and/or sensing electronics into the herein-described drug delivery systems would be within the routine skill of a person of ordinary skill in the art once such a person is provided with the information contained herein.

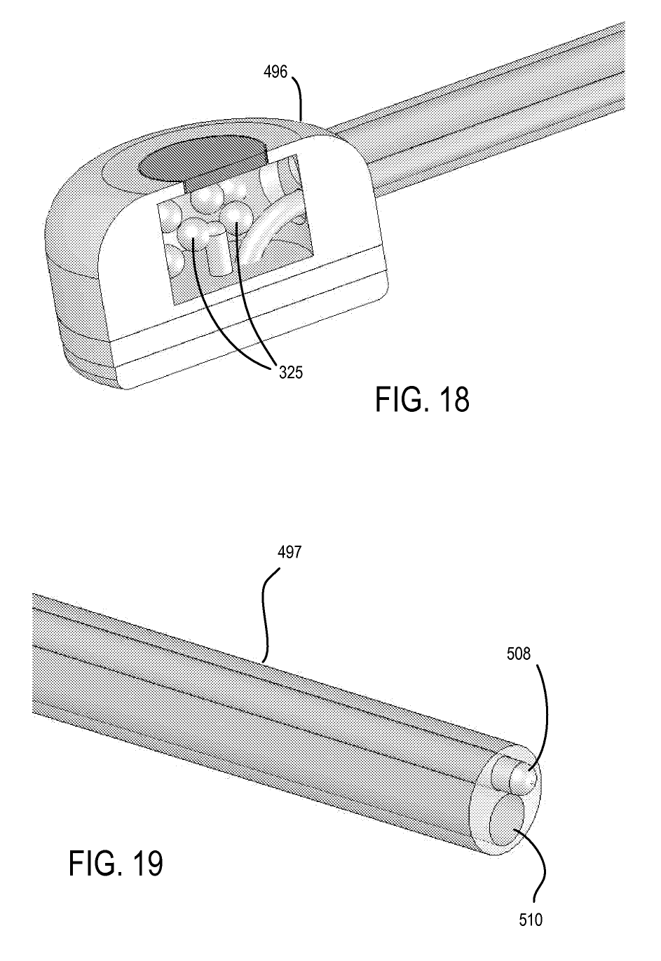

FIG. 16 shows an electrophoresis-stimulated drug delivery system 495 according to at least some embodiments. Tube 497 contains a fluid delivery lumen and an electrode wire, and extends from drug reservoir 496. FIG. 17 is a cross-sectional view of drug reservoir 496 and a portion of tube 497. Reservoir 496 includes a semi-permeable membrane 500 and an internal cavity 501 for holding solid drug pellets. An electronics package 503 and battery 505 are attached to the underside of reservoir 496. Electronics package 503 induces a charge of one polarity in electrode tip 507 and a charge of opposite polarity in a tip 508 (see FIGS. 16 and 19) of electrode wire 509. The portion of wire 509 within cavity 501 may be coated with a dielectric or otherwise insulated to prevent premature charge exchange with tip 507. FIG. 18 is similar to FIG. 17, but shows solid drug pellets 325 within cavity 501. FIG. 19 shows (in an orientation that is inverted relative to FIG. 18) the terminal (or distal) end of tubing 497 and illustrates electrode tip 508 and fluid outlet 510. When opposite charges are applied to electrode 507 and wire tip 508, an electro-osmotic flow is induced to a natural low resistance outlet within an eye. Interstitial fluid enters cavity 501 through semi-permeable membrane 500. In other embodiments, a separate tube is used (instead of membrane 500) to withdraw fluid from another bodily region that is remote from the drug reservoir. Fluid entering cavity 501 dissolves drug in cavity 501 and delivers the drug to the target ocular tissue.

In at least some embodiments, a port is subcutaneously (or transcutaneously) implanted in a patient's body and placed into fluid communication with an implanted catheter and terminal component. The port includes an internal cavity which can be used to hold liquid and/or solid drug(s). A self-sealing elastomeric (e.g., silicone) septum covers the cavity. The septum can also have a drug compatible fluoropolymer laminated lining to minimize drug adsorption. A non-coring needle may be inserted through the septum so as to introduce a fluid into the cavity from an external source. That fluid can be a liquid formulated drug, or may be a liquid vehicle for dissolving (or otherwise entraining) a solid form drug already located within the cavity and delivering that entrained drug to an eye. In some embodiments, a liquid formulated drug is used as a vehicle to entrain an additional solid-form drug contained in the cavity.

The drug-holding cavity of a port may be composed of (or coated with) a drug compatible material (e.g. stainless steel, titanium, platinum, gold or drug compatible polymer). This material may also be biocompatible (so as to prevent tissue rejection), able to withstand repeated refilling and dispensing of the drug and the potential corrosive effects of a drug-containing vehicle, and able to hold drug and remain implanted for an extended period of time without degradation. If a port is to be used for holding a drug in a solid state, the cavity-forming material may be compatible so that the drug does not stick to the cavity walls, and so that cavity surfaces coming into contact with a drug do not adsorb any of the drug. Cavity walls should not, at least in certain embodiments, be permeable to water or physiological fluids.

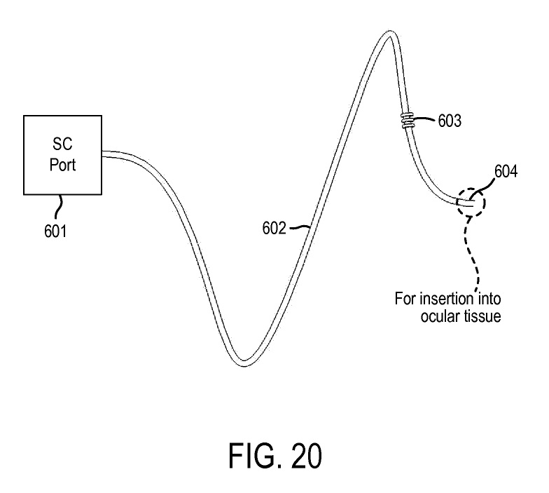

FIG. 20 shows one arrangement that includes a port. An implanted port 601 (shown in block diagram form) is connected to a catheter 602, which catheter is also implanted inside the patient's body. A terminal component 604 is located at a distal end of catheter 602. A flange or other type of stop (not shown in FIG. 20) prevents over insertion of terminal component 604 into the eye. Optional suture anchors 603 provide a means of securing catheter 602 in place. Port 601 could contain a solid drug which is then dissolved or otherwise entrained by a sterile vehicle (e.g., saline, Ringer's solution, Ringer's lactate, artificial vitreous humor and/or any other vehicle compatible with injection into the eye or other ocular tissue) introduced into the port from an external pump. Port 601 can receive drug and/or a vehicle from an external pump (e.g., the MiniMed 508 pump described in Example 2) or other external source.

In some embodiments, and as described in more detail in application 60/807,900, a subcutaneously-implantable port includes two cavities. One of those cavities is in fluid communication with a first lumen of a dual lumen catheter, and the other cavity is in fluid communication with the other lumen. Such an embodiment permits flushing of a target ocular tissue using one side of the port to receive fluid from another source (e.g., an external pump) and using the other side of the port to withdraw fluid from the target ocular tissue.

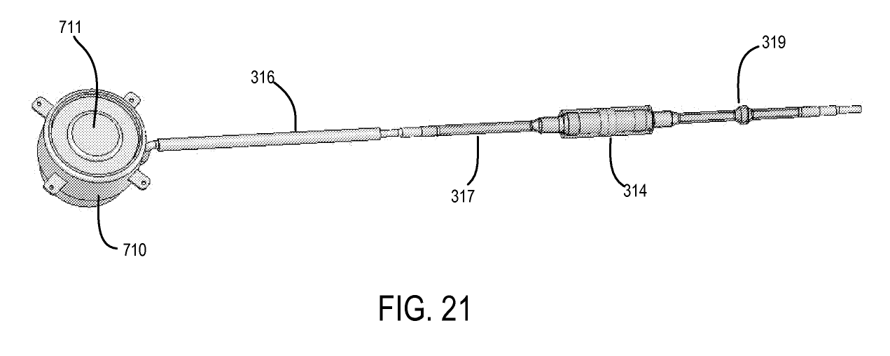

FIG. 21 shows an embodiment in which osmotic pump 312 of device 310 (FIG. 4) has been replaced with a subcutaneous port 710. In some embodiments, port 710 contains solid drug pellets which are eroded by a vehicle that is introduced into the port via a needle that pierces septum 711 of the port (with the needle in fluid communication with an external pump or some other source of vehicle).

Delivery of drug to an ocular tissue can also be performed using devices and procedures described in U.S. patent application Ser. No. 11/337,815 (filed Jan. 24, 2006 and titled “Apparatus and Method for Delivering Therapeutic and/or Other Agents to the Inner Ear and to Other Tissues,” published as U.S. Patent Application Publication No. 2006/0264897).

In some embodiments, an electronics package coupled to a drug reservoir (e.g., electronics package 503 in FIG. 17) includes components for sensing properties of a drug/vehicle solution (or suspension). The sensed properties could include one or more of pressure, absorbance of light, electrical conductivity, light scattering, drug or electrolyte concentrations, etc. These sensed properties can then be used, via appropriate electronics, to adjust operation of a pump (internal or external) or other elements (e.g., magnetic coil or electrophoretic electrodes). An electronics package could also (or alternatively) be configured to detect light or other physical parameters (e.g., tissue electrical activity) and/or be in communication with remote sensors.

In at least some additional embodiments, a vehicle used to remove drug from one or more solid drug masses in a reservoir may itself be a pre-mixed suspension of nanoparticles containing a drug (or drugs). In still other embodiments, drug devices according to various embodiments can be used to deliver a pre-mixed suspension of nanoparticles containing a drug (or drugs) without employing a solid drug mass in a reservoir chamber. In either case, the nanoparticles can be drug nanoparticles or nanoparticles of a carrier material to which drug has been absorbed/adsorbed or otherwise attached.

As previously indicated, devices and methods such as are described herein can be used to provide sustained, long term delivery of a drug. Such devices and methods can also be used to provide intermittent drug delivery on a long term basis. For example, a reservoir holding a solid drug mass could be implanted in a patient's body. That reservoir can then be periodically connected (e.g., using a subcutaneous port in fluid communication with the reservoir) to a source of vehicle.

Similar to system 310 shown in FIG. 4, the reservoirs shown in FIGS. 6A-9 can be implanted in a human or animal and coupled on one end (e.g., inlet 350 of reservoir 340, inlet barb 362 of reservoir 360) with a catheter to a vehicle source (e.g., an implanted osmotic pump, a port into which vehicle is introduced from an external source). The other end (e.g., outlet 348 of reservoir 340, barb 363 of reservoir 360) can be connected via another catheter to a terminal component implanted in an eye of a patient.

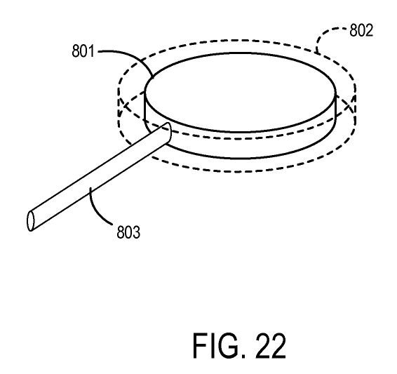

In one or more of the above-described embodiments, an ocular implant can be treated so as to include a thin film coating that includes a drug to be delivered, with that thin film coating slowly releasing the drug after placement of the ocular implant into a target ocular tissue. One example of such an ocular implant 801 is shown in FIG. 22, where the thin film coating 802 is shown with broken lines. A rod 803 or other member attached to implant 801 can be used to place implant 801 into (and remove implant 801 from) an ocular tissue. Although ocular implant 801 of FIG. 22 is a solid disk, thin film coatings can also be used with other types of ocular tissue implants (e.g., implants with electrodes for electrical stimulation and/or implants having sensors). Drugs suitable for delivery via a thin film include, but are not limited to, neuroprotective and antibiotic agents. The methods and materials that can be used to prepare drug-containing coatings are well known to those skilled in the art. An example of suitable methods and materials are those described in U.S. Pat. No. 6,627,246.

Coatings used should be both biocompatible and drug compatible. Thin films composed of bioabsorbable polymers are used in some embodiments; erosion of the film helps ensure release of the drug substance from the coating. Examples of suitable bioabsorbable elastomers are described in U.S. Pat. Nos. 5,468,253 and 6,627,246. Useful polymers include mixtures of L-lactide, D-lactide, epsilon-caprolactone, and glycolide. The relative composition of these mixtures can be used to control the rate of coating hydrolysis and adsorption, the rate of drug release, and the strength of the film. Other polymeric materials that can be used to prepare drug-releasing thin films include (but are not limited to) polyamides, polyalkylenes oxalates, poly(amino acids), copoly(ether-esters), poly(iminocarbonates), polyorthoesters, poly(anhydrides), and blends thereof. Naturally occurring polymers that can be degraded in the eye include hyaluronic acid, absorbable biocompatable polysaccharides such as chitosan or starch, fibrin, elastin, fibrinogen, collagen, and fatty acids (and esters thereof). Drug-containing polymers can be applied by spraying solutions containing dissolved polymer and drug on the surface to be coated or by dipping a portion of the implant in these solutions. Highly volatile solvents with low potential for residue or toxicity in the coating process, such as acetone, can be used in such spraying or dipping. Thin films typically provide drug delivery for a few weeks until the therapeutic in the film is exhausted. The thickness will depend on how long drug delivery is desired and the drug loading. Frequently, the thickness is 5-30 microns or less, though other thicknesses are allowed.

Coatings may be used both on implants placed within the sclera and on implants placed outside the sclera.

A 3-D filter element, such as is described above in connection with various embodiments, may be formed in various ways. As one example, a 3-D filter element can be cut or punched from a sheet of material (e.g., a biocompatible polymeric material or porous metallic material) with an appropriately small pore/channel size (such as ≦0.22 microns) for use as an anti-bacterial filter, and with the sheet having a thickness that will yield a filter element of a length that can extend along a flow path for several millimeters. The maximum pore size can be <10 microns, e.g., <2.0 microns or ≦0.22 microns. A metallic 3-D filter element can also be formed by sintering. For example, a fine metal powder such as titanium metal (with the particle diameter selected for the desired resulting pore size) can be tightly packed into a mold with the desired shape for the final filter element. The metal is heated to the point at which the powder particles begin to melt and form attachments to neighboring particles. This results in an intricate porous bonded meshwork which works like a filter, has a tortuous path and has a predetermined macro-external shape. A filter element can alternately be formed from type 316 stainless steel, porous gold, porous platinum or any other biocompatible metal. As used throughout this specification (including the claims), “metal” includes metal alloys. In certain embodiments metal alloys can be made from two materials such as gold and silver and then one metal is removed (e.g., silver dealloyed) to produce a microporous filter material.

As yet another example, micro fibers of an appropriate diameter suitable for an antibacterial filter can be incorporated into an appropriate metal and then burned out (carbon based) or etched out such as silica based ceramics (e.g., fugitive filter fibers) with hydrofluoric acid. Examples of such filter filler components are known in the art. Additional embodiments include a thin filter of the correct pore/channel size layered or laminated onto a larger porous material to provide additional strength to the thin filter. In 3-D filters prepared from polymeric material, lasers or gamma rays may be used to modify the filter materials and so as to allow etching of the pores into the filter material, producing a filter with very uniform pore diameters. Filters with larger pore size can be used together with antibacterial filters to act as a pre-filter to remove particles that may clog the antibacterial filter.

Without limitation and as further examples, a 3-D filter element (whether metallic or polymeric) can have a diameter in the range of about 0.010 inches to 0.400 inches (e.g., about 0.062 inches). The length of a 3-D filter element can be approximately 0.010 inches to 0.200 inches (e.g., about 0.039 inches). The pore size can be, e.g., ≦0.22 microns. Filter elements of other dimensions are acceptable (depending on the application and the device desired) as long as they function as an antibacterial filter; effective pore size is generally more critical than the overall dimensions, though smaller pore sizes increase back pressure.

In certain circumstances a microporous 3-D filter can be used together with an anti-bacterial thin film filter when the removal and replacement of a clogged filter is surgically convenient for the patient. For example, this could be useful when a drug port is used with an enclosed antibacterial filter as part of the assembly. Thin film membrane filters can be assembled with a supporting infrastructure to prevent liquid going around a filter. This can be done with a backing on the membrane filter and o-rings to make a liquid tight seal around the membrane edges.

A 3-D filter element (however formed) can be incorporated into a fluid system in any of a variety of ways. In addition to the incorporations described above (e.g., use of a drug/filter housing), a 3-D filter element can be inserted into a portion of a catheter or other tube (e.g., a catheter formed in part from a flexible biocompatible polymer such as silicone rubber) that is swollen (with a solvent) to allow easy insertion of the filter element into that tube. When the solvent evaporates, the tubing returns to its design diameter and closes around the filter element to make a tight seal. The outside of a 3-D filter element can also be, welded, glued or sealed with tubing to prevent leakage around the sides of the filter element.

In all of the above-described embodiments (as well as other embodiments) in which a 3-D antibacterial filter is employed, variants of those embodiments may employ a membrane filter or other type of antibacterial filter mechanism.

Although various embodiments using an implantable osmotic pump are described above, other types of implantable pumps can be used. Such other types of pumps include MEMS (microelectromechanical systems) pumps (e.g. piezo electric pumps with check valves, mini-peristaltic and other kinds of miniature pumps) containing the appropriate microfluidics.

Suture anchors can be used in many embodiments for securing a catheter and/or terminal component. Suture anchors can be molded directly to a catheter using a liquid silicone elastomer or another suitable biocompatible polymer. Suture anchors can be ring-shaped, but other shapes (e.g., squares, half-rings, thin plates or “ears” with holes for suture thread) can also be employed. Alternatively, suture anchors may be bumps on the surface of the tubing made of silicone elastomer, epoxy, or other kinds of adhesives.

Numerous types of catheters can be used in various embodiments. In at least some embodiments, implanted catheters are formed from drug- and biocompatible materials such as fluoropolymers (e.g., PTFE, FEP, ETFE and PFA), silicone rubber, PVC, PEEK, polyimide, polyethylene, polypropylene and polyurethane. The precise compound selected for a catheter will depend on the material-drug compatibility for the drug to be delivered, as well as the flexibility, lumen size and other specifications required for a particular application. Single-lumen and multi-lumen catheters can be used.

As indicated above, implantable components may be formed from (or include) a variety of biocompatible materials. Drug-contacting surfaces of components are, in at least some embodiments, formed from materials which are compatible with drugs having a pH between 4-9.

Terminal components include electrical ocular implants, and embodiments of the invention include use of an implantable drug delivery device in conjunction with, or as part of, a retinal or other intraocular electrical implant. FIGS. 23 and 24 show one example of such an embodiment. FIG. 23 is a top view of a retinal implant 901. The top of implant 901 is partially removed to reveal inner details. FIG. 24 is a cross-sectional view taken from the location indicated in FIG. 23. Specifically, retinal implant 901 includes an inner chamber 904 containing multiple electrodes 906 and fluid exit apertures 908. Attached to each electrode 906 is a conductor (e.g., a wire) 909. So as to avoid confusion, only portions of some electrode conductors are shown. Each electrode 906 extends through the bottom of implant 901 so as to have a portion exposed on lower face 911 of implant 901. In this manner, each electrode 906 is able to apply electrical stimulation to a portion of a retina with which lower face 911 is placed into contact. Apertures 908 allow fluid within chamber 904 to exit implant 901 and be delivered to the retina. Apertures could alternatively (or also) be included on a side opposite electrodes 906 so as to deliver drug to the vitreous inside the eye.

Retinal implant 901 is attached to an end of a dual lumen catheter 902. A first lumen 905 is used as a conduit to route conductors 909 from electrodes 906 to a control electronics package (not shown). A second lumen 903 is used to transport a drug containing-fluid (a liquid drug formulation, a vehicle and entrained drug, etc.) to chamber 904 for ultimate delivery to the retina. Lumen 903 may be coupled (directly or via an intervening connection catheter) to any of the implantable drug delivery devices described above. Materials for implant 901 include those described in U.S. Pat. No. 7,181,287, such as silicone or a polymer having a hardness of 50 or less on the Shore A scale, as measured with a durometer. Other materials could also be used. Electrodes 906 can similarly be formed from materials such as those described in U.S. Pat. No. 7,181,287 (e.g., platinum or an alloy thereof, iridium, iridium oxide, titanium nitride), as well as other materials. Conductors 909 could be formed from platinum, an alloy thereof or other material, and include silicone or fluoropolymer sheathes or coatings for insulation and for protection and against interaction with a drug being dispensed.

Other types and configurations of drug delivery implants can also be used, and can be used in a variety of ocular tissues (e.g., the eye, the optic nerve, the visual cortex). A drug delivery implant need not provide electrical stimulation.

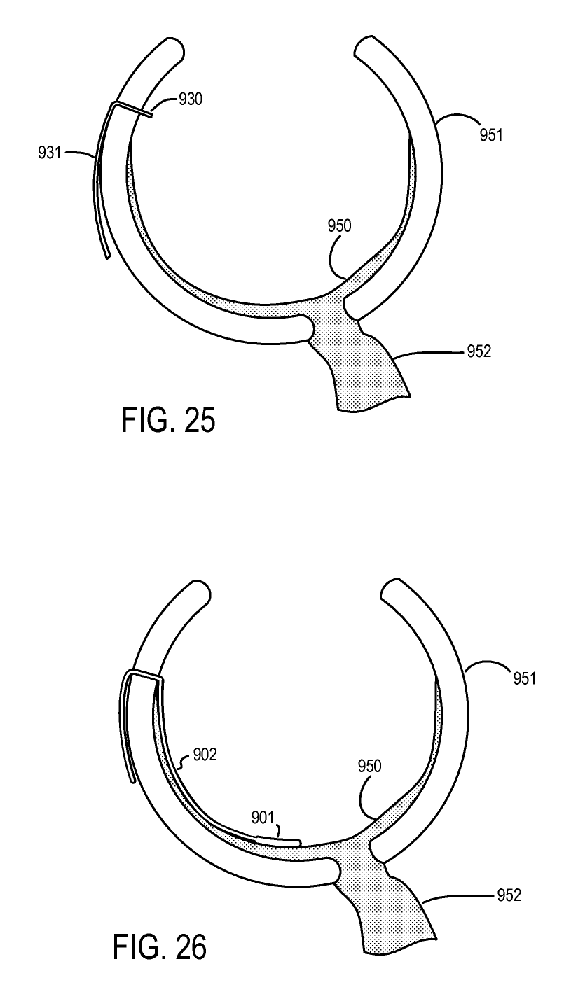

FIGS. 25 and 26 are partially schematic drawings showing placement of devices according to some embodiments within an eye (shown in cross-section) having a sclera 951, retina 950 and optic nerve 952. For simplicity, other ocular structures (e.g., lens, cornea) and tissues are omitted. FIG. 25 shows placement of a terminal component 930 (a catheter end in this case) in or near the pars plana, with terminal component 930 connected to a catheter 931. Catheter 931 is in turn connected to an implanted drug delivery device such as described previously. FIG. 26 shows placement of a retinal implant 901. An additional example of a configuration for placement of a retinal implant (together with associated electronics) is shown in U.S. Pat. No. 6,718,209.

Any of the eye conditions identified above can be treated by using one or more of the device and/or system embodiments described above. Any of the drugs described above can be delivered using one or more of the device and/or system embodiments described above. In any of the embodiments discussed above, a system could be free of filters or other components described above.

All patents and patent applications cited in the above specification are expressly incorporated by reference. However, in the event that one of said incorporated patents or applications uses a term in a manner that is different from the manner in which such term is used in the above specification, only the usage in the above specification should be considered (to the extent any language outside the claims need be considered) when construing the claims.

### EXAMPLES

The following specific examples are provided for purposes of illustration only and are not intended to limit the scope of the invention.

### Example 1

### Fabrication of Pellets of Gacyclidine Base

Water (500 mL) was brought to a boil. This hot water bath was then used to melt solid gacyclidine base. After placing 35 mg of gacyclidine base in a small glass vial, the vial was incubated in the hot water bath (90-100° C.) until the gacyclidine base melted. Small aliquots (2 μL) of the melted gacyclidine base were then transferred to polypropylene tubes (1.5 mL in size) and allowed to stand at room temperature until the gacyclidine base had solidified.

Solidification of the melted gacyclidine is typically complete within 30 minutes, but can occasionally take many hours. About half of the time, a single solid mass is obtained that slowly grows from a single focus. For those aliquots that result in multiple smaller crystalline/amorphous masses on standing, the tube containing the aliquot can be incubated in a hot water bath (90-100° C.) until it is melted a second time. Upon cooling, a second crop of single solid masses will be obtained. This process can be repeated, as necessary, until all aliquots of gacyclidine base have been converted to single solid masses.

Single solid masses (drug pellets) obtained in this way have an average weight of 1.5±0.3 mg and are hemispheres with a diameter of about 1.9 mm. These drug pellets have sufficient mechanical stability to be detached from the surface on which they are grown and transferred to a dissolution chamber.

### Example 2

### Dissolution of Gacyclidine Base in a Continuous Flow Reactor

A drug chamber similar to the one illustrated in FIG. 5 was loaded with 11 pellets of gacyclidine base having a combined mass of 18 mg. This drug-loaded chamber was eluted at a flow rate of 20 μL/hr at room temperature (23±2° C.) using a MiniMed 508 syringe pump (available from Medtronics MiniMed of Northridge, Calif.). The syringe was loaded with 3 mL of Ringer's solution containing 0.05 to 3 mM hydrochloric acid. The eluted volume was collected in PTFE tubing attached to the pump drug capsule assembly, after a 3-D antibacterial filter. The pH of this solution was determined by use of a pH meter equipped with a Calomel electrode. Drug concentration was determined by HPLC.

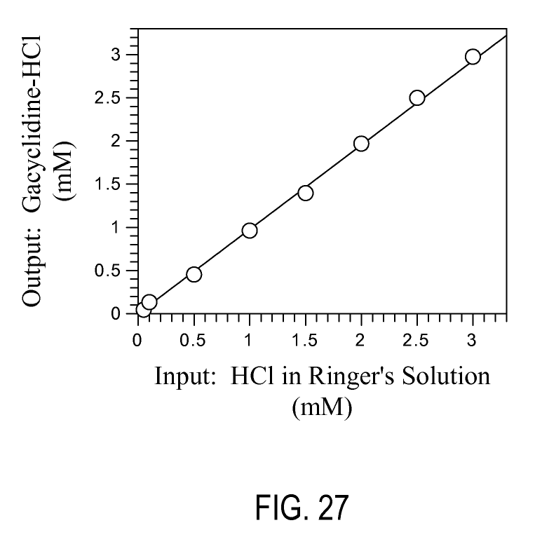

The highest pH of the eluted drug solution (5.9) was obtained at 0.05 mM hydrochloric acid, and the lowest pH of the eluted drug solution (5.6) was obtained at 3 mM hydrochloric acid. These pH values indicate quantitative conversion of the hydrochloric acid to the drug salt and are consistent with the pH expected for solutions of the hydrochloride salt. As shown in FIG. 27, the concentration of gacyclidine obtained in the output from the continuous flow reactor was linearly correlated with the concentration of hydrochloric acid used to elute the chamber. These data had a correlation of 0.976±0.049 in gacyclidine concentration per hydrochloric acid concentration used for elution and an intercept at zero concentration of hydrochloric acid of 0.0014±0.0061 mM gacyclidine.

### Example 3

### Preparation of Gacyclidine Base Pellets from Solutions of Gacyclidine Hydrochloride

Aqueous stock solutions of 1.0 M gacyclidine hydrochloride (299.9 mg/mL) and 1.0 M NaOH were prepared. Equal volumes of these solutions were mixed in a 1.7 mL polypropylene vial, then subjected to 30,000-times gravity centrifugal force in a Hermle Z229 minicentrifuge for 5 minutes. Gacyclidine base separated out during centrifugation as an oil and collected at the bottom of the centrifuge tube. Between 7 minutes and 2 hours following mixing of the solutions, the liquid gacyclidine base solidified into a single mass. The aqueous supernatants above the drug pellets were removed by aspiration by use of a sterile needle and syringe. The volumes mixed and the weights of drug pellets recovered are tabulated in Table 5.

TABLE 5

| Vol. of 1 M | Vol. of 1 M | Wt. of Drug Pellet | Wt. of Drug | |
|---|---|---|---|---|
| Gacyclidine | NaOH | Recovered | Pellet Expected | Yield |
| (μL) | (μL) | (mg) | (mg) | (%) |
| 20 | 20 | 3.2 | 6 | 53 |
| 40 | 40 | 10.3 | 12 | 86 |
| 60 | 60 | 14.2 | 18 | 79 |
| 80 | 80 | 18.9 | 24 | 79 |
| 100 | 100 | 25.4 | 30 | 85 |
| 120 | 120 | 27.3 | 36 | 76 |

### CONCLUSION

Numerous characteristics, advantages and embodiments of the invention have been described in detail in the foregoing description with reference to the accompanying drawings. However, the above description and drawings are illustrative only. The invention is not limited to the illustrated embodiments, and all embodiments of the invention need not necessarily achieve all of the advantages or purposes, or possess all characteristics, identified herein. Various changes and modifications may be effected by one skilled in the art without departing from the scope or spirit of the invention. Although example materials and dimensions have been provided, the invention is not limited to such materials or dimensions unless specifically required by the language of a claim. The elements and uses of the above-described embodiments can be rearranged and combined in manners other than specifically described above, with any and all permutations within the scope of the invention. As used herein (including the claims), “in fluid communication” means that fluid can flow from one component to another; such flow may be by way of one or more intermediate (and not specifically mentioned) other components; and such may or may not be selectively interrupted (e.g., with a valve). As also used herein (including the claims), “coupled” includes two components that are attached (movably or fixedly) by one or more intermediate components.

1. A method of delivering one or more drugs to an ocular tissue, comprising:
implanting a drug source into a human or animal;
implanting a terminal component into an ocular tissue of the human or animal, the terminal component being in fluid communication with the drug source; and
delivering one or more drugs from the drug source to the ocular tissue through the terminal component.

2. The method of claim 1, wherein implanting a drug source includes implanting a reservoir containing a mass of a solid form of the one or more drugs, and further comprising
passing vehicle from a vehicle source past the solid drug mass in the implanted drug source so as to entrain the one or more drugs from said mass, and wherein the delivering step includes delivering the vehicle and entrained one or more drugs from the terminal component.

3. The method of claim 2, wherein the vehicle source is an implantable pump.

4. The method of claim 3, wherein the vehicle source is in fluid communication with the reservoir via a catheter.

5. The method of claim 4, wherein the implantable pump is implanted in the human or animal, and wherein the delivering step includes passing the vehicle and entrained one or more drugs through an antibacterial filter implanted in the human or animal.

6. The method of claim 4, wherein the implantable pump is a MEMS pump.

7. The method of claim 4, wherein the implantable pump is a piezo-electric pump.

8. The method of claim 4, wherein the implantable pump is a piston pump.

9. The method of claim 1, wherein the delivering step includes passing a fluid containing the one or more drugs through an antibacterial filter implanted in the human or animal.

10. The method of claim 1, wherein the step of implanting a drug source comprises implanting an osmotic pump.

11. The method of claim 1, wherein the one or more drugs delivered to the ocular tissue includes at least one of the following:
gacyclidine, one of its analogs, or one of its derivatives,
an NMDA receptor antagonist,
an anti-inflammatory drug,
a steroid,
an anti-fibrotic,
an integrin antagonist,
a molecule with the RDG (Arg-Gly-Asp) tripeptide cell adhesion motif,
fibronectin,
an antibiotic,
an antisecretory molecule,
a cholinergic agent,
a neuroprotective agent,
an anti-viral factor,
an anti-angiogenic factor,
an anti-neoplastic factor, and
a neurotrophic factor.

12. The method of claim 1, wherein the delivering step includes delivery of one or more drugs to the ocular tissue for at least one of the following:
treatment of neoplastic disease,
treatment of glaucoma,
treatment of an inflammatory disease of the ocular tissue,
treatment of the ocular tissue following trauma or surgery of the ocular tissue,
treatment of the ocular tissue to prevent fibrosis following surgery or injury by infections or trauma,
treatment of the ocular tissue to prevent detached retina or another disease where cell adhesion is needed,
treatment of the ocular tissue to inhibit further retinal detachment,
treating internal infections of ocular tissue,
reducing ocular pressure from glaucoma or other disease, and
treatment of neurodegeneration.

13. The method of claim 1, wherein the delivering step includes delivery of one or more drugs to the ocular tissue for treatment of macular degeneration.

14. The method of claim 1, wherein the implanted drug source is in fluid communication with the terminal component via at least one lumen of a multilumen catheter, wherein the one or more drugs are delivered to the ocular tissue through said at least one lumen, and further comprising
relieving intraocular pressure through another lumen of the multilumen catheter.

15. The method of claim 1, wherein the implanted drug source is in fluid communication with the terminal component via at least one lumen of a multilumen catheter, wherein the one or more drugs are delivered to the ocular tissue through said at least one lumen, and further comprising
receiving fluid from the human or animal through another lumen of the multilumen catheter.

16. The method of claim 1, wherein the terminal component includes an intraocular electrical stimulator, and further comprising
providing stimulation to the ocular tissue via the intraocular electrical stimulator.

17. The method of claim 16, wherein the intraocular electrical stimulator comprises a retinal implant having a plurality of electrodes and a plurality of apertures through which the one or more drugs are delivered.

18. The method of claim 1, wherein the delivering step includes delivery of one or more drugs to the ocular tissue for prevention of neurological damage resulting from surgical implantation or other physical trauma to at least one of a structure within the eyeball, an optic nerve or a visual cortex.

19. The method of claim 1, wherein the delivering step includes delivery of one or more drugs to the ocular tissue for treatment of hyperactivity of at least one of the peripheral or central visual nervous system.

20. The method of claim 1, wherein the one or more drugs are delivered from the terminal component in a fluid that includes a suspension of at least one of
small particles 100 nm to 0.1 mm in size having an affinity for the one or more drugs being delivered, and
nanoparticles 10 nm to 100 nm in size having an affinity for the one or more drugs being delivered.

21. The method of claim 20, wherein the fluid entrains drug from a solid drug mass in the implanted solid drug source.

22. The method of claim 1, wherein the one or more drugs includes gacyclidine.

23. The method of claim 1, wherein the one or more drugs includes an NMDA receptor antagonist.

24. The method of claim 1, wherein the step of implanting the terminal component includes implanting the terminal component in an ocular tissue outside of the sclera.

25. A method of fabricating solid pellets of gacyclidine base, comprising:
neutralizing a conjugate acid form of the gacyclidine base in solution with a pharmaceutically acceptable base, and
subjecting a suspension resulting from the neutralizing step to centrifugal force.

26. The method of claim 25, further comprising:
subjecting the suspension to sterile filtration prior to the step of subjecting the suspension to centrifugal force.

27. The method of claim 25, wherein the pharmaceutically acceptable base is sodium hydroxide.

28. A method, comprising:
applying gacyclidine to an ocular tissue to treat at least one of ocular tissue trauma, macular degeneration, vein occlusion, ischemia, diabetic retinopathy, neurodegeneration, and retinal damage resulting from exposure to intense light energy.

29. A method of delivering one or more drugs to an ocular tissue, comprising:
implanting a subcutaneous port in a human or animal;
implanting a terminal component into an ocular tissue of the human or animal, the terminal component being in fluid communication with the subcutaneous port;
placing the implanted subcutaneous port into fluid communication with a pump or other fluid source located external to the human or animal; and
delivering one or more drugs from the pump or other fluid source to the ocular tissue through the implanted terminal component.

30. A method of delivering one or more drugs to an ocular tissue, comprising:
implanting in an ocular tissue an implant having a thin film coating that includes a neuroprotective agent.

31. The method of claim 30, wherein the neuroprotective agent is an NMDA receptor antagonist.

32. The method of claim 31, wherein the NMDA receptor antagonist is gacyclidine.


SEQUENCE LISTING (7 sequences)

SEQ ID NO: 1 - protein, 4 aa, Artificial Sequence
  caspase-1 inhibitor
YVAD

SEQ ID NO: 2 - protein, 6 aa, Artificial Sequence
  caspase-2 inhibitor
PVDVAD

SEQ ID NO: 3 - protein, 5 aa, Artificial Sequence
  caspase-3 inhibitor
PDEVD

SEQ ID NO: 4 - protein, 5 aa, Artificial Sequence
  caspase-4 inhibitor
PLEVD

SEQ ID NO: 5 - protein, 5 aa, Artificial Sequence
  caspase-6 inhibitor
PVEID

SEQ ID NO: 6 - protein, 5 aa, Artificial Sequence
  caspase-8 inhibitor
PDEVD

SEQ ID NO: 7 - protein, 4 aa, Artificial Sequence
  caspase-9 inhibitor
DEVD